(19) United States
(12) Patent Application Publication
(10) Pub. No.: US 2005/0272636 A1
(43) Pub. Date: Dec. 8, 2005

(54) Treatment and diagnosis of a reproductive disorder by measuring or inhibiting interferon-gamma

(75) Inventors: Sarah Anne Robertson, St. Peters (AU); David James Sharkey, Golden Grove (AU); Kelton Paul Tremellen, Stirlling (AU); Danielle J. Glynn, Hectorville (AU)

Correspondence Address: BIRCH STEWART KOLASCH & BIRCH, PO BOX 747, FALLS CHURCH, VA, 22040-0747, US

(73) Assignee: Adelaide Research & Innovation Pty Ltd., Adeaide (AU)

(21) Appl. No.: 11/082,884

(22) Filed: Mar. 18, 2005

Related U.S. Application Data
(63) Continuation of application No. PCT/AU03/01234, filed on Sep. 19, 2003.

(30) Foreign Application Priority Data
Sep. 20, 2002 (AU) 2002951531

(51) Int. Cl. A61K 39/395; A61K 38/17; A61K 38/08; A61K 31/545

(52) U.S. Cl. 514/2; 514/17; 514/192; 514/200; 424/145.1

(57) ABSTRACT

The invention provides compositions and methods for diagnosing and treating reproductive disorders. The invention also provides methods for diagnosing reproductive disorders by detecting the presence of IFN-γ in the semen of a prospective father, which is indicative of the presence of a reproductive disorder. In particular, the invention provides a method for treating a reproductive disorder in a mammal, comprising administering an effective amount of a compound which inhibits the activity of IFNγ to the mammal. The mammal may be the prospective mother or the prospective father.

This application claims priority from Australian provisional application No. 2002951531, filed Sep. 20, 2002, the contents of which are hereby incorporated by reference in their entirety.

### FIELD OF THE INVENTION

The invention provides compositions and methods for the diagnosis and treatment of reproductive disorders in mammals, particularly humans.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

An inability or reduced ability to have children can cause great personal distress and has a high attendant social cost, particularly in terms of the cost of medical intervention. A large proportion of couples fall into this category. In the USA, for example, it is estimated that some 10-15% of couples of reproductive age are unable to have children, whereas in the United Kingdom this is 14%. In 1995 it was calculated that 5.1 million women had impaired fertility in the USA alone, with this figure projected to increase to 5.9 million by the year 2020 (Stephen EH Fert Steril 66, 205-9 (1996)). In the US, the cost of a pregnancy conceived by IVF varies between US$66,000 for the first cycle to US$114,000 by the sixth cycle (Neumann et al N Eng J Med 331, 239-43 (1994)). In countries such as Australia and the United Kingdom with highly-developed systems of public health provision, assisted reproduction technologies represent a significant cost to the system.

Recent studies have revealed that a major proportion of infertile couples are childless because of a higher than normal rate of early embryonic loss (70% miscarriage v 21 % miscarriage in fertile controls; Hakim et al Am J Obstet Gyn 172, 1510-7 (1995)), rather than an inability to conceive. These findings have prompted a search for reasons for the increased rate of early embryonic loss in infertile couples, and for potential therapies to avert or minimize such losses.

In the last 20 years or so the development of in vitro fertilisation (IVF) and other assisted reproduction technologies (ART)have offered some hope to infertile couples. These IVF techniques generally take the form of stimulating the female to ovulate, contacting collected ova with sperm in vitro and introducing fertilised ova into the uterus. Multiple variations of this general process are also used, such as intracytoplasmic sperm injection (ICSI), gamete intrafallopian transfer (GIFT), and zygote intrafallopian transfer (ZIFT). Despite considerable research and technical advances in the IVF field the rate of successful pregnancy following IVF treatment is still quite low and is in the order of 15 to 25% per cycle.

Undertaking an IVF program often causes great anguish, especially when there is no resultant successful pregnancy. It is presently believed that the poor success rate in IVF treatment and other forms of ART is due to an extraordinarily high rate of early embryonic loss (Weinberg et al Fert Steril 50, 993-5 (1988), 1988, Lenton et al Ann NY Acad Sci 541, 498-509 (1988)), possibly related to the patient's impaired reproductive state or the IVF process itself. The low efficacy of IVF, together with its high cost and the associated psychological trauma from repeated treatment failures, makes alternative approaches to the problem of infertility desirable. Current methods of increasing pregnancy rates during IVF treatment include placing multiple embryos, for example 2-5 embryos, into the uterine cavity, but this is not always effective, since uterine receptivity is believed to be at fault at least as commonly as embryonic viability. Furthermore, high rates of multiple pregnancy are undesirable, since they are associated with a strongly increased maternal risk of pre-eclampsia, haemorrhage and operative delivery, and fetal risks including growth restriction and pre-term delivery, with the attendant possibility of physical and mental impairment. Children born even after singleton IVF pregnancies are now recognised to have a two and a half-fold increased risk of low birth weight (Schieve et al., New England Journal of Medicine 346, 731-737, 2002) and the risk of birth defect is reported to increase from about 4% to about 9% in such pregnancies (Hansen et al., New England Journal of Medicine 346;725-730, 2002).

Early pregnancy loss is also a major constraint in breeding programs for domestic and companion animals, livestock and rare or threatened animal species, with serious economic implications. Embryonic mortality during the pre- and peri-implantation periods is considered to be the major reason for poor pregnancy outcome when assisted reproductive technologies such as artificial insemination are used. Even following natural mating, variability in litter size and in the viability of offspring are additional limitations.

The reasons for increased rates of early embryonic loss following natural and assisted conception remain unknown. Chromosomal studies on miscarried embryos have confirmed that at least half of these embryos are genetically normal (Stem et al Am J Reprod Immun 37, 352-3 (1997 Normal embryos appear to be lost primarily because the environment provided by the maternal reproductive tract during pre-implantation development or at the time of implantation into the endometrium is insufficient to nurture their growth and development. Embryos may lose viability or developmental potential if inappropriate or insufficient nutrients or peptide growth factors are present in the maternal tract milieu . Moreover, a primary determinant of uterine receptivity is provided by the maternal immune response to the conceptus, which is perceived by the mother as foreign or semi-allogeneic as a result of expression of both maternal and paternal antigens.

Medawar originally hypothesised that maternal immune accommodation of the semi-allogeneic conceptus might be facilitated by immunological tolerance to paternal transplantation antigens, which are major histocompatibility complex (MHC) antigens (Medawar P B Symp Soc Exp Biol 44, 320-38 (1953)). This hypothesis lost favour when it was found that pregnancy does not permanently alter the capacity of mice to reject paternal skin grafts (Beer & Billingham J. Reprod. Fert. Suppl. 21, 59-88 (1974); Breyere and Burhoe Ann. NY Acad. Sci. 120, 430-434 (1964)). However, the concept of transient hyporesponsiveness to paternal MHC antigens (Breyere and Burhoe Ann. NY Acad. Sci. 120, 430-434 (1964)) is now receiving renewed attention, as a recent study by Tafuri et al. Science 270, 630-633 (1995) has provided clear evidence that during murine pregnancy, T-lymphocytes which react with paternal class I MHC become “anergic”, or unable to recognise antigen, due to internalisation of T-cell receptors. This anergic state conferred “tolerance” to paternal MHC antigen-expressing tumor cells, and was functionally operative from as early as implantation (day 4 of pregnancy) until shortly after parturition, when lymphocytes regained their reactivity. The data support the hypothesis that a permissive maternal immune response to other antigens expressed on the embryo, or on the fetal-placental unit (hereafter referred to as the conceptus) may similarly be due to induction of an immune tolerance response specific to those antigens.

Precisely what is responsible for inducing this tolerance of paternal MHC antigens and other conceptus antigens, and the nature of the tolerance, has heretofore been unclear.

Tafuri et al. (Science 270, 630-633 (1995) have shown that paternal antigen-specific tolerance is active by the onset of blastocyst implantation on day 4 of pregnancy in mice. The pre-implantation embryo is a poor antigenic stimulus, since it usually comprises fewer than 100 cells and is enveloped by a protective coat (zona pelluicida) until just before implantation. Semen, however, is richly endowed with paternal antigens present on and within sperm cells, somatic cells and the seminal plasma itself, and comprises an effective priming inoculum for many paternal antigens known to be shared by the conceptus (Beer & Billingham J. Reprod. Fert. Suppl. 21, 59-88 (1974)).

Hitherto seminal plasma has conventionally been thought to function primarily as a transport and survival medium for spermatozoa traversing the female reproductive tract (Mann: The biochemistry of semen and the male reproductive tract (John Wiley and Sons, Inc., (1964)). Recent studies by the inventors have highlighted a previously unappreciated role for semen in interacting with maternal cells to induce a cascade of cellular and molecular events which ultimately leads to maternal immune tolerance to paternal antigens present in semen and shared by the conceptus, thereby abrogating immune rejection during implantation.

Ejaculation during coitus provokes a leukocyte infiltrate at the site of semen deposition in a variety of mammalian species, including humans, which is termed the “leukocytic cell reaction” (Barratt et al. Hum. Reprod. 5, 639-648 (1990); Robertson et al., Journal for the Society for Gynecologic Investigation 9, abstract 505 (2002)). In mice, the cascade of cellular and molecular changes initiated by the introduction of semen into the uterus, in many respects, resembles a classic inflammatory response. Within hours after mating, there is a striking influx and activation of macrophages, neutrophils, and eosinophils into the endometrial stroma (De et al., J. Leukocyte Biol. 50, 252-262 (1991); Kachkache et al Biol. Reprod. 45, 860868-868 (1991); McMasteret J. Immunol. 148, 1699-1705 al (1992)), in association with upregulated expression of major histocompatibility complex (MHC) class II and CD86 antigens by endometrial dendritic cells, followed by enlargement of draining lymph nodes (Beer and Billingham, 1974 Beer & Billingham J. Reprod. Fert. Suppl. 21, 59-88 (1974); Clarke in Immunological aspects of reproduction in mammals, ed. Crighton, (Butterworths, London), pp. 153-182 (1984)). This inflammatory response is transient, and fully dissipates by the time of embryo implantation on day 4 of pregnancy (De et al., J. Leukocyte Biol. 50, 252-262 (1991); Kachkache et al Biol. Reprod. 45, 860868-868 (1991); McMasteret J. Immunol. 148, 1699-1705 al (1992)). At this stage leukocytes persisting in the endometrium are predominantly macrophages with an immunosuppressive phenotype (Hunt et al Cell. Immunol. 85, 499-510 (1984)).

The temporal changes in trafficking and phenotypic behaviour of endometrial leukocytes during the period between mating and implantation are likely to be orchestrated principally by cytokines released by steroid hormone regulated epithelial cells lining the endometrial surface and comprising the endometrial glands (Robertson et al., Crit. Rev. Immunol. 14:239-92, 1994). Of particular importance are granulocyte-macrophage colony-stimulating factor (GM-CSF) and interleukin-6 (IL-6), the synthesis of which is upregulated at least 20-fold and 200-fold respectively in estrogen-primed epithelial cells following induction by specific proteinaceous factors in seminal plasma (Robertson & Seamark Reprod. Fertil. Dev. 2, 359-368 (1990)). These factors are derived from the seminal vesicle gland (Robertson et al. J. Reprod Fert 107, 265-277 (1996)).

Previous studies have indicated that this surge in epithelial GM-CSF release is a key mediator in the post-mating inflammatory response, since injection of recombinant GM-CSF into the estrous uterus is sufficient to produce cellular changes resembling those seen following natural mating (Robertson et al., Journal of Reproductive Immunology 46: 131-154 (2000)). The inventors have found, using GM-CSF deficient mice, that the chemotactic activity of GM-CSF is likely to be compensated or augmented by an array of chemokines, the expression of which is transiently upregulated after mating (Robertson et al., Trophoblast Research 11, 101-119 (1998)), and of cytokines synthesised by activated endometrial macrophages including interleukin-1 (IL-1) and tumour necrosis factor-α (TNF-α) (McMasteret J. Immunol. 148, 1699-1705 al (1992)).

Furthermore the surge in GM-CSF release is known to facilitate embryo development and implantation through direct effects in the preimplantation embryo. The inventors have found that exposure of human (Sjöblom et al., Human Reproduction, 14: 3069-3076 (1999)) or mouse (Robertson et al., Biology of Reproduction 64: 1206-1215 (2001)) pre-implantation embryos to GM-CSF in vitro improves both their rate and extent of development and their likelihood of implantation and development into healthy progeny. These findings are supported by experiments in GM-CSF null mutant mice (Robertson et al., Biology of Reproduction, 60: 51-61 (1999)).

The nature of the seminal factors which act to stimulate GM-CSF release from the uterine epithelium has been investigated. It has been shown that the increase in uterine GM-CSF content is neither the result of introduction of GM-CSF contained within the ejaculate, nor a consequence of a neuroendocrine response to cervical stimulation, and is independent both of the presence of sperm in the ejaculate and MHC disparity between the male and female (Robertson & Seamark Reprod. Fertil. Dev. 2, 359-368 (1990)). A mechanism involving induction of GM-CSF mRNA synthesis in epithelial cells by proteinaceous factors derived from the seminal vesicle was suggested by experiments showing that seminal vesicle-deficient (SV-) males did not evoke GM-CSF release or a post-mating inflammation-like response in females, and that trypsin-sensitive, high molecular weight material extracted from the seminal vesicle could upregulate GM-CSF release from uterine epithelial cells in vitro (Robertson et al. J Reprod Fert 107, 265-277 (1996)). One seminal factor that was found to have a bearing on fertility conditions is TGFβ, and we have shown that administration of TGFβ can induce GM-CSF expression, activate the ‘leukocytic cell reaction’ and lead to tolerance of paternal antigens and an improved fertility outcome (WO 98/39021, the contents of which are hereby incorporated by reference in their entirety) (Tremellen et al., Biology of Reproduction, 58, 1217-1225 (1998)).

IFN-γ is a multifunctional cytokine synthesised by activated natural killer cells and T-lymphocytes that plays important roles in inducing and modulating immune responses (Boehm et al., Annu. Rev. Immunol. 15, 749-795 (1997)). IFN-γ and IL-2 are the cytokines associated with a Th1 immune response are (Gafter et al 1997). IFN-γ antagonises the type 2 and Th3 skewing properties of TGFβ, acting to prevent TGFβ signalling through transcriptional induction of the inhibitory SMAD-like decoy, SMAD-7 (Ulloa et al., Nature. 397(6721):710-3 (1999)).

IFN-γ has been implicated in infertility. For example, maternal blood levels of IFN-γ and secretion of IFN-γ by activated peripheral blood cells tended to be higher in instances of recurrent miscarriage (RM) than in normal pregnancy (Raghupathy et al. 1999, Jenkins et al 2000; Paradisi et al 1996; Shaarwy et al. 1997; Naz et al 1995; Rezaei et al 2002), and TGF-β levels were concomitantly lower (Fornari et al 2002). Generally a decrease in the production of the type 1 cytokines IL-2 and IFN-γ by peripheral blood mononuclear cells, accompanied by an increase in the type 2 cytokines IL-4 and IL-10, is observed in normal pregnancies (Marzi et al 1996).

Injection of either anti-IFN-γ antibodies or pentoxifyllin, an anti-TNF agent, in a pregnant female partially reduced the incidence of fetal resorption, and that intraperitoneal administration of IFN-γ can induce Th1 cytokines and cause abortion (Chaouat et al., Journal of Immunology 1995). Other studies also appeared to show that administration of IFN-γ to pregnant females can cause abortions (Clark et al., 2000; Athanassakis et al. 2000). All of these studies used administration of IFNγ at the time of implantation or after embryo implantation.

Similarly, intraperitoneal administration of low doses of recombinant IFN-γ to pregnant females was shown to increase the incidence of abortion. (Vassiliadis et al 1994). It has also been shown that IFN-γ induces the expression of class II MHC(Ia) antigens on the placenta and causes fetal death via maternal immune rejection of the fetus.. In another study, spontaneous abortion in a mouse model was promoted by injecting TNF-α and IFN-γ, but Th1 cytokine-triggered abortion appeared to be dependent on the availability of lipopolysaccharide (Clark et al., AJRI, 2002; see also Sikka et al., 2001; Athanassakis et al 2000). Conversely, however, production of IFN-γ by stimulated peripheral blood mononuclear cells has been reported to be lower in one cohort of pregnant women previously experiencing recurrent pregnancy loss (Bates et al 2002), and appropriate expression of IFNγ in the implantation site can be essential for normal placental development in mice (Ashkar et al., J Exp Med. 192:259-70, 2000).

Previous studies have shown the presence of cytokines such as TNF-α and IFN-γ in the semen of infertile men. These data suggest, however, that the common inflammatory cytokines TNF-α and IFN-γ have only limited detrimental effects on sperm motility and viability, and that this may contribute to the poor fertilizing potential of human spermatozoa. Moreover both TNF-α and IFN-γ were required for these actions. In addition, it was shown that high IFN-γ levels in seminal plasma levels is associated with poor sperm viability (Estrada et al., 1997). In another study, IFNγ had no impact alone but was found to synergise with lipopolysaccharide to reduce sperm motility, viability and membrane integrity (Sikka et al, Int J Androl. 24:136-41, 2001). Other studies have shown that IFN-γ is present in seminal plasma (Maegawa et al 2002). In all cases tested, there was no indication that the partners of any of the men suffered from recurrent miscarriage (Paradisi et al. 1996). In another study, IFN-γ levels in seminal plasma were not significantly different between fertile and immunoinfertile couples (Naz et al. 1994).

Recurrent abortion in some women has been shown to be associated with production of toxic factor(s) by the embryo- and/or trophoblast in response to stimulation by sperm or trophoblast antigens, and that the principal factor may be IFN-y, which was shown to mediate embryotoxicity (Hill et al 1992). IFN-γ has been shown to induce specific proteins on trophoblasts, and these proteins are responsible for embryotoxic antibody production in the mother (Athanassakis et al 1996).

It has been suggested that coitus-induced IFN-γ production by peripheral blood lymphocytes in women sensitized to sperm may impair fertility and early embryo development by a mechanism other than a direct effect of anti-sperm antibodies on the male gamete (Witkin et al 1989). IFN-γ and IL-4 secreting cells were found to be significantly higher in the blood of an RM group compared with controls, both before and during pregnancy, but it was concluded that production by cells in the reproductive tract may have a greater bearing (Palfi et al 1999). Other studies in mice suggested that spontaneously increased decidual IFN-γ expression is detrimental to embryo survival, and that IFN-γ is the primary signal for macrophage activation that leads to early embryo loss (Haddad et al 1997). TNF-α and IFN-γ can exert deleterious effects in the placenta, and tend to be present in low concentrations, whereas the regulatory cytokines IL-10 and TGF-β are beneficial, and tend to predominate (Entrican et al 2002).

In sum, although it has been reported that IFN-γ in females may have detrimental effects on pregnancy, little is known about the source of the IFN-γ or about whether reduction in IFN-γ levels can improve fertility and prevent a reproductive disorder. Moreover, although the presence of IFN-γ in seminal plasma of infertile men has been reported, this has been associated with poor sperm viability, and there has been no suggestion that this IFN-γ may have deleterious effects on the induction of immune tolerance to paternal antigen, which is required for successful pregnancy.

It is apparent, therefore, that new and improved methods for improving fertility are greatly to be desired. It is particularly apparent that methods for reducing or preventing recurrent miscarriage are highly desirable.

### SUMMARY OF THE INVENTION

It is therefore an object of the invention to provide new methods for diagnosing reproductive disorders. It is a further object of the invention to provide compositions and methods for treating reproductive disorders.

In accordance with these objects, a first aspect of the invention provides a method of treating a reproductive disorder in a mammal, comprising the step of administering an effective amount of a compound which inhibits the activity of IFN-γ to the mammal. The mammal may be a human, and it will be clearly understood that either the prospective mother or the prospective father is healed. In a specific embodiment, the compound may be a compound, such as an antibody, that binds to IFN-γ and/or the IFN-γ receptor. The antibody may be a human antibody. The compound may also be a soluble IFN-γ receptor, a β-lactam antibiotic, a protamine, a highly charged peptide, a highly charged protein, and/or a peptide comprising the sequence Arg-Arg-Lys-Trp-Gln.

In any of the foregoing methods, the prospective mother may also be administered an effective amount of a TGF-β protein, for example, TGFβ1, TGFβ2, TGFβ3, activin or an analogue thereof.

In any of the foregoing methods, the prospective mother may also be administered an effective amount of a GM-CSF.

In any of the foregoing methods, the prospective mother may also be exposed to one or more antigens of a prospective father. This exposure may occur on a plurality of occasions prior to treatment with the IFN-γ inhibitor.

In accordance with a second aspect of the invention, there is provided a method of treating a reproductive disorder in a mammalian prospective mother, comprising assaying the semen of a prospective father for the presence of IFN-γ and, if IFN-γ is detected, administering to the prospective mother an effective amount of a composition that inhibits the activity of IFN-γ. The prospective mother may also be exposed to one or more antigens of the prospective father on one or more occasions prior to, contemporaneously with, and/or after treatment with the IFN-γ inhibitor.

In any of the foregoing methods, the IFN-γ inhibitor may be administered at the same time the prospective mother is exposed to the semen of the prospective father.

In any of the foregoing methods the IFN-γ inhibitor may be administered to the prospective mother prior to conception, and may be administered systemically and/or via a mucosal surface of the mother.

In any of the foregoing methods the IFN-γ inhibitor may be administered systemically to a prospective father prior to delivery of semen from the father to the prospective mother.

In any of the foregoing methods, the semen of a prospective father may be treated with a compound which inhibits the activity of IFN-γ prior to delivery of the semen to the prospective mother.

In accordance with a third aspect of the invention there is provided a method of treating a reproductive disorder in a mammalian prospective mother, comprising the step of administering to the prospective mother an effective amount of a TGF-β, where the TGF-β is not TGFβ1, TGFβ2, TGFβ3, activin or an analogue thereof.

In accordance with a fourth aspect of the invention there is provided a method of treating a reproductive disorder in a mammalian prospective mother, comprising the step of assaying the semen of a prospective father for the presence of IFN-γ and, if IFN-γ is detected, administering to the prospective mother an effective amount of a TGF-β, for example, TGFβ1, TGFβ2, TGFβ3, activin or an analogue thereof.

The IFN-γ is preferably detected by ELISA, and the detectable level of IFN-γ is preferably equal to or greater than about 1 pg/ml, more preferably equal to or greater than about 2 pg/ml, more preferably equal to or greater than about 3 pg/ml, more preferably equal to or greater than about 4 pg/ml, more preferably equal to or greater than about 5 pg/ml, more preferably equal to or greater than about 6 pg/ml, more preferably equal to or greater than about 7 pg/ml and even more preferably equal to or greater than about 8 pg/ml. Other, more sensitive assay methods are known in the art.

Preferably the IFN-γ level is higher than the level present in semen of a control sample. Preferably the control sample is semen from a male partner of a female who does not suffer from a reproductive disorder and, in particular, does not suffer from a reproductive disorder caused by the lack of immune tolerance to paternal antigen.

The TGF-β may be administered via a mucosal surface. The prospective mother may also be exposed to one or more antigens of the prospective father; for example, the antigens may be sperm antigens or MHC class I antigens present on leukocytes or in seminal plasma of the prospective father, or a derivative or analogue of such an antigen, which comprises an epitope of the antigen. The antigens of the prospective father may be administered to the mother via a mucosal surface. The mother may be exposed to the antigens of the prospective father before, during or after treatment with the TGFβ. The TGFβ may be administered to the prospective mother prior to conception. Preferably the method of treating a reproductive disorder induces immune tolerance to said one or more antigens. The mother may in addition be administered an effective amount of a compound which inhibits the activity of IFN-γ.

In accordance with a fifth aspect of the invention, there is provided a method of diagnosing a reproductive disorder in a mammalian prospective father, comprising the step of comparing the amount of detectable IFN-γ in a semen sample obtained from the prospective father with a control sample from a male who does not suffer from such a disorder, where a IFN-γ value higher than the control sample is indicative of the reproductive disorder. Preferably the control sample is semen of a male partner of a female who does not suffer from a reproductive disorder and, in particular, does not suffer from a reproductive disorder caused by the lack of immune tolerance to paternal antigen. The IFN-γ is preferably detected by ELISA, and the detectable level of IFN-γ is preferably equal to or greater than about 1 pg/ml, more preferably equal to or greater than about 2 pg/ml, even more preferably equal to or greater than about 3 pg/ml, more preferably equal to or greater than about 4 pg/ml, still more preferably equal to or greater than about 5 pg/ml, particularly more preferably equal to or greater than about 6 pg/ml, more preferably equal to or greater than about 7 pg/ml and most preferably equal to or greater than about 8 pg/ml. Other, more sensitive assay methods are known in the art.

According to a sixth aspect, the invention provides a composition for treatment of a reproductive disorder, comprising semen of a prospective father, together with
(a) an inhibitor of IFN-γ, and/or
(b) a member of the TGF-β family,
and optionally also comprising a pharmaceutically-acceptable carrier.

According to a seventh aspect, the invention provides a composition for treatment of a reproductive disorder, comprising
(a) a sperm or MHC Class I antigen of a prospective father,
(b) an inhibitor of IFN-γ, and/or
(c) a member of the TGF-β family, and
   a pharmaceutically-acceptable carrier.

In either of these two aspects, the composition is preferably a vaginal cream, tampon or pessary.

In an eighth aspect, the invention provides the use of a compound which inhibits the activity of IFN-γ for the manufacture of a medicament for the treatment of a reproductive disorder in a mammal. The use may be in conjunction with use of a TGFP which is not TGFP-1, TGFP-2, TGFP-3, activin or an analogue thereof, GM-CSF, or a sperm antigen or MHC Class I antigen present on leukocytes or in seminal plasma of a prospective father or a derivative or analogue of such an antigen which comprises an epitope of the antigen.

In any of the foregoing aspects of the invention, the reproductive disorder may be recurrent miscarriage, miscarriage, spontaneous abortion, pre-eclampsia, early embryonic loss, subfertility, or implantation failure.

In any of the foregoing aspects of the invention, the reproductive disorder may be caused by lack of immune tolerance to paternal antigen. For example, the lack of immune tolerance may be caused by the type 1 immune-deviating properties of IFN-γ, particularly IFN-γ present in the semen of the prospective father.

In any of the foregoing aspects of the invention the IFN-γ inhibitor, the TGFβ, the GM-CSF or the antigens of the prospective father may be administered in the form of a composition comprising the respective active agent together with a pharmaceutically-acceptable carrier.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

It will be clearly understood that the methods and compositions of the invention may be used in conjunction with conception via intercourse, or in conjunction with assisted reproduction technologies, including but not limited to artificial insemination, IVF, (ICSI), GIFT or ZIFT.

While it is particularly contemplated that the methods and compositions of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

### BRIEF DESCRIPTION OF THE DRAWINGS

FIG. 1 shows the IFN-γ content of seminal plasma of men classified according to fertility status. Symbols represent data from individual men. The cut-off value for quantifiable IFN-γ of 5 pg/ml is shown with a horizontal line.

FIG. 2A is a histogram showing the effect of varying the concentration of IFNγ on GM-CSF production in UEC cells and reversal of that effect at certain concentrations of the IFNγ inhibitor penicillin.

FIG. 2B is a histogram showing the effect of varying the concentration of IFNγ on GM-CSF production in UEC cells, where GM-CSF production is stimulated by recombinant TGFP.

FIG. 3 shows the effect of TGFβ₁ and IFNγ on GM-CSF synthesis in murine uterine epithelial cells. The GM-CSF content of culture supernatants was determined by bioassay (A, C and D) or commercial ELISA (B, E & F), in supernatants collected 24 h after 16 h incubation with rmIFNγ alone or in combination with TGFβ₁ (A), TGFβ₂ (B) TGFβ₃ (C); αIFNγ antibodies (E) or αIFNγR1 antibodies (F). In one experiment cells were incubated with nmIFNγ in the presence and absence of penicillin (60 mg/ml)(D).

FIG. 4 is a histogram showing the effect of varying the concentration of IFN-γ on GM-CSF production in human cervical epithelial cells, where GM-CSF production is stimulated by recombinant TGFβ. The GM-CSF content of culture supernatants was determined by commercial ELISA, 12 h after the addition of rTGFβ₁ or rIFNγ alone or in combination.

FIG. 5 shows expression of RNAs encoding IFN-γR and TGFβR2 in murine uterine epithelial cells. (A) Gel electrophoresis of RT-PCR demonstrating IFN-γR and TGFβR2 PCR amplicons in whole uterine tissue. (B) Quantitative RT-PCR analysis showing abundance of IFN-γR mRNA transcripts in Arbitrary Units in two preparations of whole uterine tissue (WUT1 and WUT2), purified uterine epithelial cells (UEC) and residual uterine stromal cells (RS). Expression in liver and kidney is shown as a positive control. Circles represent data from two or three replicate experiments and mean values are scored. (C) Quantitative RT-PCR analysis showing abundance of TGFβR2 mRNA transcripts in Arbitrary Units in two preparations of whole uterine tissue (WUT1 and WUT2), purified uterine epithelial cells (UEC) and residual uterine stromal cells (RS). Expression in liver and kidney is shown as a positive control. Circles represent data from two or three replicate experiments and mean values are scored.

FIG. 6 shows the results of a quantitative RT-PCR analysis showing variance in abundance of IFN-γ mRNA transcripts in Arbitrary Units in n=17 preparations of human cervical tissue, where the mean value was attributed a value of 100 Arbitrary Units. Symbols represent data from individual subjects normalised to β-actin mRNA content and the median value is scored.

FIG. 7 shows the effect of IFN-γ on uterine luminal fluid content of GM-CSF on day 1 of pregnancy in mice. Mice received 5 ng rmIFN-γ (n=5), 20 ng rmIFN-γ (n=6) or PBS-BSA alone (n=5). Symbols represent data from individual mice and the median value is scored. *p=0.03, PBS control group versus IFN-γ treatment groups combined.

### DETAILED DESCRIPTION

In the context of this specification the term “reproductive disorder” is to be understood to encompass not only the capacity to conceive, but also recurrent miscarriage (RM), miscarriage, spontaneous abortion and other pregnancy-related conditions, such as pre-eclampsia, early embryonic loss, and implantation failure, and includes subfertility. It will be clearly understood that a reproductive disorder may result from factors affecting the prospective mother, the prospective father, or both, and that a reproductive disorder may result from a combination of factors which is specific to the individual couple. A “reproductive disorder” is to be understood to encompass both infertility conditions and gestational disorders, where infertility means inability to establish a viable pregnancy as measured by detection of hCG in the maternal blood.

The term “tolerance” in this specification is taken to mean inhibition of the potentially destructive cell-mediated immune response against conceptus or paternal antigens, and/or inhibition of synthesis of conceptus antigen-reactive immunoglobulin of complement-fixing isotypes, for example the “type 1” or “Th1” compartment of the immune response. This tolerance may or may not be associated with induction of synthesis of non-destructive, conceptus antigen-reactive immunoglobulin of non-complement-fixing isotypes and subclasses, for example the “type 2′ or “Th2′ compartment of the immune response, or with recruitment into the implantation site of lymphocyte subsets with regulatory or suppressor activity, for example “Th3” lymphocytes. The term “tolerance” encompasses T cell anergy and other permanent or transient forms of hyporesponsiveness or suppression of the maternal type 1 compartment. The term “couple” means a prospective mother-father pair, and is intended to encompass both human and other mammalian pairs.

The term “IFN-γ inhibitor” means a compound or agent which is able to reduce or abolish the activity of IFN-γ, and includes an agent which interferes with the physiological activity of IFN-γ by any mechanism, including preventing the binding of IFN-γ to a cellular receptor, preventing the synthesis of an IFN-γ cellular receptor, or preventing the synthesis of IFN-γ, as well as by directly binding to IFN-γ itself.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids. Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

In the claims of this application and in the description of the invention, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

The present inventors have discovered that IFN-γ in the semen of the male partner of a couple suffering from RM is a cause of, and diagnostic for, the RM condition in many couples. The presence of IFN-γ in the semen promotes the development of a Th1 type immune response, leading to immune-mediated rejection of the implanted foetus and suppresses the ability of TGF-γ to induce GM-CSF expression and the development of a Th2 type immune response.

The invention provides methods for diagnosing the existence of a reproductive disorder in a couple by detecting the presence of IFN-γ in the semen in the male partner of the couple and/or by measuring the level of IFN-γ in the semen. The invention also provides methods for treating a reproductive disorder by antagonizing, inhibiting, or the activity of IFN-γ in one or both partners of an infertile couple. The inhibition of IFN-γ in either partner of the couple may be systemic, and/or may be local. In particular, a composition that inhibits IFN-γ may be used to inhibit seminal IFN-γ activity in the semen by, for example, adding the composition directly to the semen, and/or by topical administration to the genital tract of the female partner.

The present inventors have discovered that TGF-β in the semen acts to induce GM-CSF activity in the female partner, and thereby to induce a desired Th2 type (tolerance) immune response in the female. The IFN-γ acts to antagonize seminal TGF-β activity and prevent occurrence of the Th2 response. The Th2 response can therefore be restored not only by inhibiting IFN-γ activity directly, for example by use of a composition that binds to IFN-γ or blocks the IFN-γ receptor or that systemically inhibits IFN-γ expression, but also by overcoming the IFN-γ-induced inhibition of GM-CSF synthesis by administration of supplemental TGF-β and/or GM-CSF. The present invention therefore also comprehends methods of treating a reproductive disorder in a couple by first assaying for the presence of IFN-γ, or the presence of elevated levels of IFN-γ, in the semen of the prospective father, and thereafter treating the infertility condition by administration of supplemental TGF-β and/or GM-CSF. The TGF-β protein may be one or more of the TGF-β family of proteins, as described in more detail below.

Demonstration of IFN-γ Activity in Semen and Correlation with Infertility

In an extreme form, a reproductive disorder can result from prevention of conception by a lack of immune tolerance for sperm cells. In the majority of cases of reproductive disorders, however, conception occurs normally, but a lack of immune tolerance to the implanted foetus causes spontaneous abortion of the foetus.

Previous studies have indicated that unidentified factors in components of the ejaculate can indirectly contribute to pregnancy success, as shown by experiments in accessory gland-deficient mice (Pang et al. J. Reprod. Fert. 56, 129-132 (1979); Peitz & Olds Clarke. Biol. Reprod. 35, 608-617 (1986)) and the finding that poor pregnancy outcome and dysregulated fetal and/or placental growth after embryo transfer or during first pregnancy in various livestock species (Polge. Control of pig reproduction, eds. Cole & Foxcroft. (Butterworths, London), pp. 277-291 (1982); Mah et al J. Anim. Sci. 60, 1052-1054 (1985); Walker et al. Theriogenology 37, 111-126 (1992)) can be partially ameliorated by prior exposure to semen (Murray et al. J Anim Sci 56, 895-900 (1983); Stone et al. Proc. Am. Fert. Soc. 43, 88 (1987)). Likewise, studies in humans have now clearly identified an association between lack of exposure to semen due to limited sexual experience, use of barrier methods of contraception, or in IVF pregnancies and increased risk of implantation failure, spontaneous abortion and pre-eclampsia (Klonoff-Cohen et al. JAMA. 262, 3143-3147 (1989); Robillard et al The Lancet 344, 973-975 (1995); Bellinge et al. Fertil. Steril. 46, 2523-2526 (1986)). Conversely, exposure to semen can improve implantation success after embryo transfer during IVF treatment (Tremellen et al., Human Reproduction 15, 2653-2658(2000)).

As described above, the presence of IFN-γ in semen of infertile men has previously been observed, but there has been no suggestion that this cytokine was associated with a reproductive disorder, in particular one associated with lack of immune tolerance to paternal antigen. The present inventors have measured the levels of IFN-γ in the semen of prospective fathers and correlated the level of IFN-γ with the occurrence of RM, and found that the presence of, or the presence of an elevated level of, IFN-γ correlates strongly with the occurrence of RM. Semen from prospective fathers was analysed using an ELISA specific for human IFN-γ. The results showed that the presence of IFN-γ in the semen correlated with the occurrence of RM in the couple.

Although IFN-γ may occasionally be found in the semen of the male of a fertile (non-RM) couple, it is present at low levels, and elevated levels of IFN-γ are not observed. In particular, in the context of the present invention, levels of IFN-γ equal to or higher than about 5 pg/ml of semen are usually predictive of the likelihood of RM. Accordingly, the invention provides a diagnostic method for assessing the prospects for a positive fertility outcome by ascertaining the level of IFN-γ in the seminal fluid of a prospective father.

It is known that the relative level of TGFP in seminal fluid is a positive indicator of a fertility outcome, whereas the present invention demonstrates that IFN-γ is a negative indicator. It is also possible, therefore, to assess the prospects for a positive fertility outcome by measuring the levels of TGF-β and/or IFN-γ in the seminal fluid of a prospective father. In particular, observation of the relative levels of TGF-β and IFN-γ may be used to predict a prospective fertility outcome. More particularly, a reduced level of TGF-β concomitantly with the presence of IFN-γ, and in particular an elevated level of IFN-γ, is strongly predictive of a negative fertility outcome. Either cytokine can be measured by methods that are well known in the art, for example, by ELISA or other forms of immunoassay. Methods and instruments for measuring IFN-γ and TGF-β levels in biological fluids are commercially available and well known in the art. It is also possible to assess the likelihood of a positive fertility outcome by measuring the ability of a sample of seminal fluid to stimulate the production of GM-CSF in allogeneic cervical epithelial cells in vitro. This can be achieved by contacting a sample of the cells with a sample of seminal fluid, and measuring GM-CSF production, using methods that are well known in the art.

The likely fertility outcome can be assessed by measuring IFN-γ levels in a prospective mother, either instead of, or in addition to, measurement of IFN-γ and/or TGF-β levels in the seminal fluid of a prospective father. Measurement of IFN-γ levels in a prospective mother can be carried out in any suitable tissue or bodily fluid sample from the mother. For example, measurement of serum levels of IFN-γ will give an indication of IFN-γ levels in the reproductive tract, although IFN-γ levels in the reproductive tract may also be measured, for example in vaginal or cervical secretions or in uterine washings, or in tissue samples obtained by biopsy. The measured level of IFN-γ can then be compared to a reference level, for example, a reference level from a fertile female or collection of fertile females, and the relative level determined. The reference level can be corrected for demographic variation and the like, if necessary, using methods that are well known in the art.

In all methods of the present invention, levels of protein expression, and in particular cytokine expression, may be measured at the protein or nucleic acid level. For example, measurement of protein expression at the protein level can be performed by immunoassays such as ELISA (direct and sandwich), immunohistochemistry, Western blot, radioimmunoassay, flow cytometry and neutralization, although the skilled artisan will be aware of other methods that are well known and available. Suitable immunoassay methods are described, for example, in U.S. Pat. No 4,666,865, the contents of which are incorporated herein by reference in their entirety. Suitable reagents and kits to perform these tests are available from R&D Systems Inc., Minnesota (www.rndsystems.com). Suitable reagents and kits available from R&D Systems Inc., to measure human IFN-γ include anti-human IFN-γ antibody (Cat. No. AF-285-NA); biotinylated anti-human IFN-γ antibody (Cat. No. BAF285); DuoSet ELISA for human IFN-γ (Cat. No. DY285); human IFN-γ development module (Cat. No. SEL285); ELISpot for human IFN-γ (Cat. No. EL285); Carboxyfluorescein conjugated IFN-γ monoclonal antibody (Cat. No. IC285F); Fluorokine MAP human IFN-γ kit (Cat. No. LU285); Monoclonal anti-human IFN-γ antibody Cat. No. MAB285); Phycoerythrin-conjugated monoclonal IFN-γ antibody (Cat. No. IC285P); Quantkine human IFN-γ ELISA kit (Cat. No. DIF50); anti-human IFN-γ polyclonal antibody (Cat. No. AB 250 NA) and affinity purified anti-human IFN-γ polyclonal antibody (Cat. No. AF 250 NA). A radioimmunoassay kit for human IFN-γ is available from Celltech, Berkshire, England. Alternatively, mass spectrometry, chromatography (including high pressure liquid chromatography), gel electrophoresis and biological activity assays can be used to measure IFN-γ levels and activity in human samples.

Measurement of protein expression at the nucleic acid level can be achieved by quantitative RT-PCR methods, PCR-ELISA, and in situ hybridization techniques that are well known the art. Suitable reagents and kits available from R&D Systems Inc., to measure human IFN-γ mRNA expression include human IFN-gamma Quantikine mRNA kit (Cat. No. KRNIF0-0); human IFN-gamma primer pair (Cat. No. RDP-14-025); Quantikine mRNA Probes and Calibrator Kit for human IFN-γ (Cat. No. RNIF0-036). Alternatively, microarray-based methods and the like may be used. Such microarray methods are commercially available, for example from Affymetrix (Sunnyvale, Calif.). Although absolute levels of protein expression can be measured, it is also possible to measure relative levels of protein expression using methods that are well known in the art. For example, protein levels can be measured compared to an internal or external reference sample, or can be compared to levels in a suitable database containing information about relative levels of protein expression. Both the reference levels and database information may be corrected, if desired, for demographic variables such as ethnicity, age, etc. Internal reference samples can be obtained from a tissue or fluid that is remote from the cells involved in semen production (in males), or that are remote from the reproductive tract (in females). External reference samples can be from individuals with a history of successful fertility. Database reference figures may be obtained in similar fashion.

Methods of Treating a Reproductive Disorder

The invention also provides compositions and methods for treating a reproductive disorder by overcoming the deleterious effect of IFN-γ that is present either in the semen of the prospective father and/or in the reproductive tract of the prospective mother. In particular, the invention provides compositions and methods for promoting a tolerant (Th2) immune response in the mother by, for example, promoting GM-CSF production in the maternal reproductive tract.

IFN-γ in the semen of the prospective father or in the reproductive tract of the prospective mother antagonizes the activity of TGF-β present in the seminal fluid. Accordingly, this antagonistic effect can be overcome by a variety of methods, and the skilled artisan will recognize that the present invention comprehends any method by which the deleterious activity of IFN-γ is wholly or partially overcome, and is not limited to the specific methods exemplified herein. In addition, the specific methods described herein may be used individually or in combination, and/or may be used with other methods that presently are known or that are described in the future for inhibiting the effects of IFN-γ.

As broadly described, the present invention provides methods for treating a reproductive disorder, and more specifically an RM condition, in a human or other mammal by exposure of a prospective mother to an IFN-γ inhibitor before, during, or after attempted conception, thereby eliciting GM-CSF synthesis or a transient hyporesponsive immune reaction to one or more antigens of a prospective father thereby to alleviate symptoms of the infertility condition. The exposure to the IFN-γ inhibitor can occur in combination with exposure of a prospective mother to one or more antigens of a prospective father. Both the exposure to the IFN-γ inhibitor and the exposure to the antigen(s) can independently occur before, during or after attempted conception.

In addition to inhibition of IFN-γ the deleterious activity of IFN-γ also can be overcome by use of a protein that overcomes the antagonism of TGF-β activity by the IFN-γ. More specifically, in one method of achieving this result, the expectant mother is exposed to a pharmaceutical composition comprising a protein having TGF-β-like activity. In particular, proteins and protein fragments of the TGF-β family may be used. These proteins and protein fragments include, but are not limited to, TGFβ₁, TGFβ2, TGFβ₃, inhibin, Mullerian Inhibiting Substance (MIS) and activin. These proteins are described in more detail below. The protein may be used alone or in combination, or in combination with IFN-γ inhibitors of the type described above.

IFN-γ Inhibitors

IFN-γ inhibitors include antibodies that specifically bind IFN-γ or the cellular receptor(s) for IFN-γ activity, soluble IFN-γ receptor or fragments thereof, and drugs that inhibit IFN-γ activity.

IFN-γ inhibitors include drugs and peptides which suppress the synthesis of IFN-γ, such as neuropeptide hormones or peptides which are immunoreactive with neuropeptide hormones. The regulation of IFN-γ by adrenocorticotropic hormone (ACTH) and a peptide which is immunoreactive with ACTH has been discussed by Johnson et al., J, Immunol. 132, 246 (1984). The regulation of IFN-γ by thyrotropin has been discussed by Chung et al. (Endocrinology 141:2090-2097, 2000).

IFN-γ inhibitors also include molecules, for example, peptides, which prevent or inhibit the interaction of IFN-γ with a cellular receptor involved in the directing the immune response. Examples of this type of antagonist are described below.

Also included are antibodies to IFN-γ, and antibodies to the IFN-γ cellular receptor. These antibodies may be polyclonal ormonoclonal, and are described in more detail below. Monoclonal antibodies are preferred.

A soluble form or fragment of the IFN-γ receptor may also be a very potent form of inhibitor. Forms of IFN-γ receptor and fragments thereof are described in U.S. Pat. No. 5,578,707, and recombinant versions are described in U.S. Pat. No. 5,763,210, the contents of which are herein incorporated by reference in their entirety.

Other known categories of IFN-γ inhibitors include highly charged peptides and proteins, including protamines and salts thereof and highly charged homo- or heteropolypeptides containing lysine and/or arginine or glutamic acid and/or aspartic acid. In addition, polypeptides comprising the amino acid subsequence Arg-Arg-Lys-Trp-Gln are known to inhibit IFN-γ activity. These compounds are described in more detail below.

It will be clearly understood that because the level of TGFβ is also important in treatment of a fertility condition, the present invention may also encompass the combination of an IFN-γ inhibitor with TGFβ.

Anti-IFN-γ and Anti-IFN-γ Receptor Antibodies

IFN-γ activity can be inhibited using antibodies that bind to IFN-γ and block binding to its receptor, or that directly bind to the receptor and block binding of the cytokine. For example, these antibodies may be used in vivo in the prospective mother and/or father, or may be used in vitro by addition to a sample of seminal fluid from the prospective father. In vivo administration may be topical and/or systemic.

Polyclonal antibody preparations have been raised in rabbits in response to administration of a synthetic peptide corresponding to the first 20 amino acids of the amino (N) terminal of IFN-γ. (Johnson, et aI., J. Immunology, 129, pp.2357-2359 (1982)). Anti-IFN-γ antibodies blocking various biological activities of native IFN-γ (often referred to as “neutralizing antibodies”) are known in the art, and are disclosed in Billiau et al., Immunol. Today 9, 37-40 (1988); Hereman et al, J. Exp. Med. 171, 1853-1859 (1990); Landolfo et al., Science 229, 176-179 (1985); Didlake et al., Transplantation 45, 222-223 (1988), Jacob et al., J. Exp. Med. 166. 789-803 (1987); Yong. et al, Natl. Acad. Sci. USA 88: 7016-7020 (1991). See also U.S. Pat. Nos. 4,599,306, 4,666,865, and 4,897,264, the contents of which are hereby incorporated by reference in their entirety. Monoclonal antibodies that specifically bind the IFN-γ receptor may also be used.

The monoclonal antibody may be a chimeric molecule incorporating light and heavy chain regions from different species, and which is expressed using recombinant DNA methods. See, for example, Tan et al, J. Immunol. 135, 3564 (1985). An example of this type of antibody contains the hypervariable regions from non-human antibodies inserted into human V_{H} and/or V_{L} framework sequences. Other types of recombinant antibodies are described in more detail below.

Antibodies directed against IFN-γ may be made by any of the known techniques, using IFN-65 , or immunogenic peptides of IFN-γ, as the immunogen. IFN-γ used as the immunogen may be synthesized naturally, e.g., by induction of peripheral blood lymphocytes by phytohaemaglutinin and phorbol myristic acetate, and purified. A procedure for the induction of human IFN-γ and its purification has been described by Yip et al. Science 215, 411 (1982). Alternatively, IFN-γ or its immunogenic peptides may be synthesized by recombinant techniques. Recombinant IFN-γ is commercially available. See, e.g., Zlotnik et al. J. Immunol. 131, 2814 (1983). Immunogenic peptides of IFN-γ may also also be chemically synthesized. In instances in which the synthesized peptide is correctly configured so as to provide the correct epitope, but is too small to be immunogenic, the peptide may be linked to a suitable carrier to form a conjugate.

If polyclonal antibodies are desired, a suitable mammal, such as a mouse, rabbit, goat, horse, etc., is immunized with IFN-γ or its immunogenic peptide or conjugate. Serum from the immunized animal is collected and treated according to known procedures. If serum containing polyclonal antibodies to IFN-γ contains antibodies to other antigens, the IFN-γ can be purified by immunoaffinity chromatography, using methods well known in the art. See,for example, Antibodies; A Laboratory Manual, Harlow and Lane (Eds)(CSH Press).

The general methodology for making monoclonal antibodies via hybridoma technology is well known. Monoclonal antibodies directed against IFN-γ may be made from antibody-expressing hybridomas by procedures such as those described by Kohler and Milstein Nature 356, 497 (1975), Levy and Dilley, Proc. Natl. Acad. Sci. U.S.A. 75. 4211 (1978); and Ollson and Kaplan, Proc. Natl. Acad. Sci., U.S.A. 77, 5429-5431; and Goding: Monoclonal Antibodies: Principles and Practice (Academic Press, 3^{rd} ed. 1996).

Although xenogeneic antibodies may be used in the invention, it is preferable to use antibodies from thae same spesies as the one to be treated in orderto reduce the likelihood of the antibodies themselves inducing an immune response. In a particular embodiment of the invention, a human antibody is used. Methods for making fully human antibodies include the use of phage display techniques from a large human antibody library, as described in U.S. Pat. No. 5,969,108, which is incorporated herein by reference in its entirety. Other methods for making fully human antibodies include the use of so-called “xenomouse” technology, using transgenic mice that encode a large portion of the human antibody repertoire. These methods are provided commercially, for example by, Abgenix (Fremont Calif.) and Medarex (Princeton N.J.). See also, U.S. Pat. No. 6,075,181; Lonberg, “Transgenic Approaches to Human Monoclonal Antibodies.” Handbook of Experimental Pharmacology 113 (1994): 49-101; Lonberg et al.,. “Human Antibodies from Transgenic Mice.” Internal Review of Immunology 13 (1995): 65-93. Other methods for making human antibodies are well known in the art.

Alternatively, humanized antibodies in which CDR regions from a murine anti-IFN antibody are grafted into a human antibody framework may be used. See Riechmann et al., Nature 332:323-327 (1988). See also U.S. Pat. No. 5,585,089, which is herein incorporated by reference in its entirety.

The antibodies may be prepared in one or more immunoglobulin classes (IgM, IgG, IgA, IgD, or IgE), depending upon the particular reproductive disorder and individual involved. Antigen binding fragments of IgG monoclonal antibodies, such as F(ab′)₂, Fab, Fab′, or Fv, may also be used in appropriate situations, for instance, where it is desired to reduce the likelihood of complement fixation. Recombinant antibody fragments, such as scFv and Fab fragments may be used using methods that are well known in the art.

Antibodies to a native IFN-,y receptor which inhibit the binding of native IFN-γ to its receptor and thereby block IFN-γ biological activity are disclosed in EP 369,413; EP 393,502; EP 416,652; EP 240,975; and U.S. Pat. No. 4,897,264 issued Jan. 30, 1990. Other methods for making allogeneic antibodies against IFN-γ receptor molecules are well known in the art, and include all the methods described above for preparing anti-IFN-γ antibodies.

IFN-γ Receptor Binding Moieties and Soluble IFN-γ Receptor Moieties

Compounds that bind to the IFN-γ receptor may be used to inhibit IFN-γ activity. Thus, the inhibitor may be a peptide or peptides which mimic the tertiary conformation of IFN-γ and thereby are either competitive, noncompetitive, or uncompetitive inhibitors of IFN-γ with respect to receptor binding. Such peptides may be identified, for example, using phage display methods where suitable peptides are selected from a large peptide library by selection against a solid surface upon which the ligand binding domain of the IFN-γ receptor is immobilized. Methods for carrying out such phage display selections are well known in the art. Reagents for peptide phage display are available from, for example, New England Biolabs, Beverley, Mass. Once binding peptides have been identified, their properties may further be assessed, for example by competitive binding assays with IFN-γ where the ability of the peptide, or a mixture of peptides, to block or inhibit IFN binding to its receptor is determined. Such assays are well known in the art.

The IFN-γ inhibitor may also be an IFN-γ receptor that nonproductively binds to the cytokine i.e. that does not activate an immune response signalling pathway upon binding. Suitable receptors have been purified from different human (Aguet et al., J. Exp. Med. 165, 988-999 (1987); Novick, et al. J. Biol. Chem. 262, 8483-8487 (1987); Calderon et al., Proc. Natl. Acad. Sci. USA 85, 4837-4841 (1988)) and murine (Basu, et al., Proc. Natl. Acad. Sci. USA 85, 6282-6286 (1988)) cell types, and have been characterized as 90- to 95-kDa single chain integral membrane glycoproteins that display certain structural heterogeneity due to cell specific glycosylation. The primary sequence of the human and murine IFN-γ receptors is known and the receptors have been cloned and expressed. See Aguet, supra, and Gray et al., Proc. Natl. Acad. Sci. USA 86:8497-8501 (1989). These receptors are membrane glycoproteins, but the skilled artisan will recognize that the extracellular, non-membrane bound form of the receptor will be soluble, and can also be used in the present invention. Ligand binding domains of the receptor have been identified, as described beow.

In a particular embodiment, the IFN-γ inhibitor comprises the extracellular domain of an IFN-γ receptor, optionally fused to a stable plasma protein. The stable plasma protein is preferably an immunoglobulin, and the fusion preferably comprises at least a hinge region and the CH2 and CH3 domains of an immunoglobulin heavy chain.

In another aspect, the invention provides a bispecific molecule comprising an IFN-γ inhibitor amino acid sequence and a further amino acid sequence capable of binding a target involved in the initiation or development of a gestational disorder. As described above, the IFN-γ inhibitor may be an IFN-γ receptor, an anti-IFN-γ antibody, an anti-IFN-γ receptor antibody and an IFN-γ variant, and the further amino acid sequence is preferably from an IFN-γ inhibitor having a different specificity, an IL-1 inhibitor, a TNF-α inhibitor, a CD11a/18 inhibitor, a CD11b/18 (VLA-4) inhibitor, or an L-selectin inhibitor. The two amino acid sequences preferably are covalently linked, for example as a fusion protein. Methods for preparing nucleic acid sequences encoding such proteins are well known in the art. For example, fusion proteins consisting of the mouse IFN-γ receptor extracellular portion and constant domains of immunoglobulin molecules have been made and proposed to be useful in the therapy for autoimmune diseases, chronic inflammation, delayed type of hypersensitivity and allograft rejection. See Kurschner et al., J. Biol. Chem. 267., 9354-9360 (1992); Dembic et al., J. of Interferon Research. 12, suppl. Sep. 1, 1992].

In an alternative embodiment, an IFN-γ variant can be used that can bind to the IFN-γ receptor without activating the relevant signalling pathway. Such molecules are known in the art and typically contain a receptor binding domain but lack the domains necessary for signal transduction. Such molecules are described in U.S. Pat. Nos. 6,558,661 and 5,108.901, and in European Publication No. 146,354, the contents of which are hereby incorporated by reference in their entirety. Receptor binding domains in IFN-γ from various species, such as human and mouse, have been identified (see, for example, Lord, et al, Mol. Immunol. 26, 637-640 (1989); Favre, et al., Mol. Immunol. 26, 17-25 (1989); Jarpe et al, J. Immunol. 3304-3309 (1990); Magazine et al., Biochemistry 30, 5784-5789 (1991)).

In yet another embodiment, IFN-γ activity may be inhibited using a so-called immunoadhesin, as described, for example, in U.S. Pat. Nos. 6,558,661 and 5,116,964, EP 355,068, and PCT application WO 91/05871, the contents of which are hereby incorporated by reference in their entirety. Immunoglobulins and certain variants thereof that are suitable for immunoadhesin preparation are known, and many have been prepared in recombinant cell culture. For example, see U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Nat'l. Acad. Sci. USA 77:2197 (1980); Raso et al., Cancer Res. 41.2073′(1981); Morrison et al., Ann. Rev. Immunol. 2:239 (1984); Morrison, Science 229:1202 (1985); Morrison et al., Proc. Nat'l. Acad. Sci. USA 81:6851 (1984); EP 255,694; EP 266,663; and WO 88/03559. Reassorted immunoglobulin chains also are known (see for example U.S. Pat. No. 4,444,878; WO 88/03565; and EP 68,763 and references cited therein), as are synthetic antibody binding sites (Fv analogues) produced by protein engineering [see e.g. Huston, J. S. et al., Proc. Natl. Acad. Sci. USA 85, 5879-5883 (1988), and U.S. Pat. No. 5,091,513.

Other IFN-γ Inhibitors

As described above, highly charged peptides and proteins can be used to inhibit IFN-γ activity. Protamines are strongly basic proteins of relatively low molecular weight which are associated with nucleic acids,, and can be obtained in large quantities from ripe sperm cells of fish. Examples of protamines include salmine from salmon, clupeine from herring, and sturine from sturgeon sperm. Although salmine has been used below for purposes of illustration, all protamines can be used in the methods of this invention.

The protamines are available commercially or can readily be prepared using known methods. Salmine can be purchased, e.g., from Sigma Chemical Co., St. Louis, Mo. Because of their highly basic character, protamines are frequently provided as chloride, phosphate, sulfate or other salts. Protamine sulfate was used in an example below.

Another category of inhibitors is exemplified by poly(D-lysine), poly(L-lysine) and poly(L-glutamic acid). Although homopolymers may be used, heteropolymers containing both lysine and arginine or both glutamic acid and aspartic acid, in random or predetermined order, can be used as well. Both D and L isomers of the amino acid residues can be used, or mixtures of the two. Size is not critical. Polypeptides of molecular weight ranging from about 3,000 to about 100,000 daltons or more can be used.

A wide variety of homo- and heteropolypeptides are available commercially from sources such as Sigma Chemical Co., St. Louis, Mo. Alternatively, they can be synthesized using standard methods, and fractionated by size using gel filtration or other methods.

Testing of IFN-γ Inhibitors

The ability of an IFN-γ inhibitor to inhibit IFN activity can be measured using any in vivo or in vitro assay accepted in the art, including, but not limited to, inhibition of virus replication in a suitable cell line (e.g. inhibition of encephalomyocarditis virus replication in human lung carcinoma cell line A549 for human IFNγ), induction of MHC class II antigens, heat lability, other antiviral, antitumor or immunoregulatory assays, or neutralization by antibodies having immunoreactivity for IFN-γ but not IFN-α or -β.

For example, the ability of an inhibitor to block the induction of expression of specific antigens by IFN-γ can be assayed, essentially as described in Example 2 of U.S. Pat. No. 6,558,661 (IFN-γ induction of gene expression assay). In another group of assays (antiviral assays), the ability of an inhibitor candidate to block the protective effect of IFN-γ against viral infection is tested. A specific antiviral assay is disclosed in Example 2 of U.S. Pat. No. 6,558,661 (IFN-γ antiviral activity assay).Alternatively or in addition, a host immune-response model may be used to test the ability of an IFN-γ inhibitor to block endogenous IFN-γ, for example as disclosed in Example 2 of U.S. Pat. No. 6,558,661 (Murine listeriosis experiment).The same example also discloses a suitable mouse model for testing IFN-γ inhibitor action.

The ability of an inhibitor to bind IFN-γ (such as in the case of IFN-γ receptor or anti-IFN-γ antibodies) can also be tested by equilibrium binding analysis, essentially as described by Ashkenazi et al., Proc. Natl. Acad. Sci. USA 88, 10535-10539 (1991). According to this method, the IFN-γ inhibitor candidate is immobilized on to microtiter wells coated with goat anti-human IgG Fc antibody, and is incubated with detectably labeled IFN-γ. A similar method is described in Examples 5B and 5C of EP 393,502. Alternatively or in addition, the affinity of binding can be tested in competition binding assays, such asthose disclosed by Fountoulakis et al., J. Biol. Chem. 265, 13268-13275 (1990). These methods also can be used to measure the ability of an IFN-γ inhibitor to inhibit IFN-γ binding to its receptor in an analogous manner.

In another example, the ability of the IFN-γ inhibitor to inhibit IFN-γ can be measured by assaying the ability of the inhibitor to block the ability of IFN-γ to suppress the production of GM-CSF in murine uterine epithelial cells. A suitable assay is described, for example, in Example 2 of this specification.

TGF-β Family Proteins

As described above, supplementation of TGF-β levels can be used to overcome the deleterious effects of IFN-γ in prospective parents. More specifically, administration of supplemental TGF-β can be used when, for example, the presence of IFN-γ or elevated IFN-γ is detected in the semen of a prospective father. A variety of members of the TGF-γ family of proteins can be used for this purpose, either alone or in combination.

In mammalian species the TGFβ family comprises at least three closely related polypeptides, TGFβ₁, TGFβ₂ and TGFβ₃ (Massague. (1990) Annu. Rev. Cell Biol. 6, 597-641) which exhibit 70-80% sequence homology and share many biological actions. TGFβ₁, TGFβ₂, TGFβ₃ and activin have been identified as capable of eliciting an increase in uterine GM-CSF and, accordingly, each protein is suitable for use in the present invention. Each of these proteins may also be administered as a complex with a suitable carrier protein, such as the 250-300 kDa binding protein betaglycan (Andres. (1989) J. Cell Biol. 109, 3137-3145).

In a preferred form, the TGFβ polypeptide used for the treatment of an reproductive disorder is selected from the group consisting of TGFβ₁, TGFβ₂, TGFβ₃, TGFβ4, TGFβ5 and activin (including activin A activin B and activin AB). More preferably, the TGFβ polypeptide is selected from the group consisting of TGFβ₁, TGFβ₂, TGFβ₃ or activin (including activin A activin B and activin AB). The TGFβ polypeptide to be used is preferably derived form the same species as the subject to be treated;however, it will be understood that TGFβ polypeptides from other species may be used. For example bovine TGFβ polypeptide may be used in a human. The protein is preferably human TGFβ₃ having the amino acid sequence shown below:

TABLE 1
human TGF33 DNA and amino acid seciuence

Amino acid sequence
ALDTNYCFRN LEENCCVRPL YIDFRQDLGW KWVHEPKGYY
ANFCSGPCPY LRSADTTHST VLGLYNTLNP EASASPCCVP
QDLEPLTILY YVGRTPKVEQ LSNMVVKSCK CS
Nucleic acid sequence
GCT TTG GAC ACC AAT TAC TGC TTC CGC AAC TTG GAG GAG AAC TGC TGT
GTG CGC CCC CTC TAC ATT GAC TTC CGA CAG GAT CTG GGC TGG AAG TGG
GTC CAT GAA CCT AAG GGC TAC TAT GCC AAC TTC TGC TCA GGC CCT TGC
CCA TAC CTC CGC AGT GCA GAC ACA ACC CAC AGC ACG GTG CTG GGA CTG
TAC AAC ACT CTG AAC CCT GAA GCA TCT GCC TCG CCT TGC TGC GTG CCC
CAG GAC CTG GAG CCC CTG ACC ATC CTG TAC TAT GTT GGG AGG ACC CCC
AAA GTG GAG CAG CTC TCC AAC ATG GTG GTG AAG TCT TGT AAA TGT AGC
TGA

This TGFβ is available from GroPep Ltd (Thebarton, South Australia).

Alternatively, another member of the TGFβ superfamily may be used in the present invention. For example, a polypeptide selected from the group consisting of Mullerian inhibitory substance (MIS), bone morphogenetic proteins (BMP-2-7), inhibins, growth differentiation factor (GDF-1), dorsalin-1 (dsl-1) and Drosophila decapentaplegic gene product (DPP-C) may be used. Other peptides may be assessed for their suitability for use in the methods of the present invention, for example by assaying their ability to induce GM-CSF expression in cultured cervical epithelial cells as described below. Preferably the TGFP polypeptide member of the TGFβ family contains an intact cystine knot.

It will also be understood that various modifications can be made to TGFβ₁, TGFβ₂, TGFβ₃, TGFβ4, TGFβ5 or activin which may be effective in improving the stability or bioavailability of the molecule while retaining its ability to elicit an effective transient tolerant immune reaction, either separately or in combination with another agent. Such modified TGFβs include substitution, deletion or addition mutants and include peptide fragments, which may or may not be incorporated into another protein to make a recombinant protein. Alternatively, other polypeptide members of the TGFβ superfamily may also be used or used as a starting point to developing an analogue of the TGFβ activity, including Mullerian inhibitory substance (MIS), bone morphogenetic proteins (BMP-2-7), growth differentiation factors (GDF-1), dorsalin-1 (dsl-1) and Drosophila decapentaplegic gene product. The present invention also extends to the use of biologically active fragments, functional analogues and derivatives of TGFβ, i.e. fragments, analogues or derivatives of TGFβ, in which the wild-type TGFβ sequence contains additions, deletions or substitutions by other amino acids or amino acid analogues, in which the biological activity of the TGFβ is retained. Methods to identify, manufacture and characterise biologically active fragments, functional analogues or derivatives of TGFβ are well known to those of ordinary skill in the art. See for example Tuan et al (Connective Tissue Res 34(1): 1-9, 1996) and Lyons et al (J Cell Biol 11 0(4):1361-7, 1990). In particular, methods for preparing such fragments, analogues and derivatives are known in the art, and these compounds can be assessed for the ability to stimulate GM-CSF synthesis in cervical epithelial cells using the methods described below. The modification used to prepare such fragments, analogues and derivatives preferably should not alter the tertiary structure of the cystine knot, and preferably does not alter the TGFβ receptor binding site.

In a preferred form the TGFβ fragment, functional analogue or derivative has at least 70% amino acid sequence identity with a native TGFβ amino acid sequence, or preferably at least 90%, more preferably 95%. Methods for assessing amino acid sequence identity are well known in the art, and can be addressed by no more than routine experimentation. For example, a suitable program for determining percentage sequence identity is BLAST 2.0 Sequence Comparison (NIH) (http://www.ncbi.nlm.nih.gov/blast/b12seq/b12.html). Other suitable programs are commercially available. Preferably the limiting parameters imposed for determining sequence identity to take into account gaps, inserts, conservative substitutions and the like in a particular program are the default settings for the program, for example the default parameters shown for the BLAST program as displayed on the NCBI web site.

The invention also contemplates the use of one or more TGFβ proteins in which the coding sequence for the polypeptide is fused in-frame to a polypeptide sequence which aids in expression of the fusion protein from a host cell. A non-limiting example of such a leader sequence is the polypeptide leader sequence encoding a fragment of pig growth hormone as described in U.S. Pat. No. 5,330,971, the contents of which are herein incorporated by reference in their entirety. Other suitable leader sequences are known in the art.

The TGFβ may be administered in its active form; however, where the prospective mother is capable of activating TGFβ it may also be administered in its precursor form. Preferably the TGFβ is the TGFβ homologue specific to each species. The TGFβ may be isolated from a naturally-occurring source, or it may be chemically synthesized or produced by recombinant DNA technology; preferably the TGFβ is recombinant TGFβ. Methods of manufacturing TGFβ by recombinant DNA techniques are well known to those of ordinary skill in the art, and can be addressed with no more than routine experimentation. For example the TGFβ may be manufactured by recombinant DNA technology as described in U.S. Pat. No. 6,425,769, the contents of which are herein incorporated by reference in their entirety. The TGFβ is preferably at least about 70% pure, more preferably at least about 90% pure, most preferably at least about 95% pure, although the skilled artisan will recognize that other purities may effectively be used.

Pharmaceutical Compositions

The TGF-β proteins, IFN-γ inhibitors and other compositions of the present invention, are usually administered as pharmaceutical compositions, usually formulated in dosage forms by methods known in the art. Methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

Suitable pharmaceutical compositions comprising IFN-γ receptor amino acid sequences and antagonist anti-IFN-γ receptor antibodies and suitable dosages and dose rates are disclosed in EP 369,413; EP 393,502; EP 416,652; EP 240,975; and U.S. Pat. No. 4,897,264. The formulation of IFN-γ inhibitors is preferably a liquid or a gel, and may be a physiological salt solution or dextrose solution, together with one or more conventional stabilizers and/or excipients. IFN-γ compositions may also be provided as lyophilized powders. IFN-containing pharmaceutical compositions are disclosed in U.S. Pat. Nos. 4,727,138, 4,762,791, 4,925,793, 4,929,553, and 4,855,238.

Pharmaceutical compositions containing members of the TGF-β family are known in the art and are described, for example, in WO 98/39021, which is herein incorporated by reference in its entirety. Preferably the TGF-β is formulated in a hydroxypropyl methylcellulose gel for intravaginal administration. Even more preferably the TGF-β formulated in a hydroxy propyl methylcellulose gel is administered by use of a intravaginal applicator. The level of TGF-β may be varied, and will vary depending upon which species is being treated. For humans the concentration of TGF-β will preferably be greater than 50 ng/ml with a total dose of 150 ng/ml, and more preferably between 100 and 400 ng/ml with a total dose between 100 and 2000 ng. The level of TGF-β in normal male semen is in the order of 200 ng/ml. Alternatively, the level of TGF-β will preferably be at a concentration of between 200 ng/ml and 125 μg/ml with a total dose between 100 ng and 625 μg. Even more preferably, the level of the TGF-β will be at a concentration selected from the group consisting of 200 ng/ml, 1 μg/ml, 5 μg/ml, 25 μg/ml and 125 μg/ml.

Methods of Administering Compositions Comprising IFN-γ Inhibitors and TGF-β Family Proteins

An IFN-γ inhibitor may be administered systemically to either male or female subjects, for example by oral administration (where suitable) or intravenous injection. In a male subject, such delivery would be expected to diminish the IFN-γ content of seminal fluid. In a female subject, when paternal antigen is also to be administered, systemic delivery may occur before, during or after delivery of antigen.

In a particular embodiment of the invention, the IFN-γ inhibitor or composition comprising the inhibitor, may be topically administered to the reproductive tract of the prospective mother, using methods that are well known in the art. In addition, or alternatively, when paternal semen is administered by artificial insemination, the inhibitor may be added to the seminal fluid prior to insemination. Administration of a member of the TGF-β family can be achieved using similar routes. When paternal antigen is also to be administered, it may be desirable to deliver the IFN-γ inhibitor and/or TGF-β family member and the antigen together, for example where the molecules are combined in a gel, or spray. Alternatively, it may be desirable to provide a source of IFN-γ inhibitor and/or TGF-β family member at the mucosal surface of interest, which might be the genital tract, and the antigen could subsequently be deposited onto the mucosal surface. Although it is preferred for the IFN-γ inhibitor and/or TGF-β protein to be present at the same time as the antigen is present, it is also possible to have a delay between the delivery of the IFN-γ inhibitor/TGF-β protein and the surface antigen. Thus an alternative method would be to deposit the antigen first, perhaps as an ejaculate, and then deliver the IFN-γ inhibitor and/or TGF-β protein as a pessary after intercourse. Of course, delivery of the inhibitor/protein could also occur prior to intercourse.

An alternative delivery option is to provide “natural” TGFβ, for example in the form of platelets. Thus instead of purified or recombinant TGFβ a preparation of platelets or other source rich in natural TGFβ, such as milk or colostrum, may be used. For example the TGFβ may be contained in an extract purified from cheese whey in sufficient quantities to elicit the biological activity of the TGFβ. Suitable methods for obtaining extracts exhibiting biological activities of TGFβ include those described in United States patents U.S. Pat. Nos. 5,866,418 and 6,194,208. The TGF β may be in a substantially purified form, and preferably is at least 70% pure, more preferably at least about 90% pure, most preferably at least about 95% pure, although the skilled artisan will recognize that other purities may effectively be used.

Preferably a mucosal surface of the prospective mother is exposed to the antigen, and more preferably the mucosal surface is the genital mucosal surface.; However, exposure at other mucosal surfaces can also give rise to the transient paternal antigen tolerance. Thus it is known that tolerance to external antigens can be elicited at mucosal surfaces, and that women who are exposed orally to seminal fluid show evidence of reduced pre-eclampsia effects in response to MHC antigens of the male partner (Koelman et al J. Reprod. Immunol. 46, 155-66. (2000)). Thus the antigen exposure could be oral, respiratory, gastrointestinal or genital. For example the antigen (and optionally IFN-γ inhibitor and/TGF-β protein) may be presented as an oral or nasal spray, or as a rectal or vaginal gel, or in an enteric-coated formulation suitable for delivery of the active agent to the small or large intestine.

Whilst a mucosal exposure is preferred because it is most likely to give rise to a transient tolerant immune reaction, it is also feasible to provide for another route of exposure. Thus the surface antigen and IFN-γ inhibitor and/or TGF-β protein may be injected for systemic contact.

The surface antigens used in the present invention are preferably antigens that are particularly prominent either on the sperm or on the conceptus. Preferably these antigens are MHC antigens, and more preferably MHC class I antigens. These antigens may be presented on any appropriate cell of the intended male parent that expresses them, including sperm cells or leukocytes. The antigens may also be presented in a biological fluid such as seminal plasma, which is known to carry certain male antigens (Kajina et al Am J Reprod. Immun. 17, 91-95). Cells other than sperm cells may be used where the sperm count of the prospective father is low. Cells other than sperm cells may be preferred where a non-genital route is used. Alternatively the antigens may be presented in purified or semi-purified form, optionally presented on inert or adjuvant carriers, such as as ISCOMS. It is also contemplated that the antigens may be encoded within sperm cells in the form of mRNA (or other nucleic acid); this RNA message is then expressed by maternal genital tract cells.

The level of exposure to antigens may vary. In a preferred form the exposure will be to the prospective mother's genital tract in the form of the prospective father's ejaculate, and the level of exposure will be determined by the cell count and antigenic density on the surface of the cells. Where cells are administered other than in this manner, a similar number of cells may be used, however, the most effective manner may be determined empirically in each case. However, it is contemplated that an exposure of leukocytes in the order of 10⁷-10⁹ cells is an appropriate level of exposure to a mucosal surface.

The exposure is preferably a multiple exposure, preferably performed over a period of days or weeks, and more preferably at least three months, with the mucosal surface being exposed to IFN-γ inhibitor and/or TGF-β protein during each exposure to the prospective father's antigens.

When a male subject is to be treated, it is envisaged that the IFN-γ inhibitor would be administered over a period of time, preferably over a period of three months, during which time female contact with male antigens would occur by intercourse.

This period of time in both male and female subjects could however be somewhat reduced, and it is possible to achieve improvement with one exposure. However, exposure for at least one week is preferred before conception is attempted. Preferably where the antigens are associated with sperm cells and these are administered to the genital tract non-barrier contraceptive measures are taken prior to the planned conception,, so that there is some certainty of a period of exposure to the prospective father's antigens before conception. This is particularly the case where the reproductive disorder is of the type where conception takes place but is followed by either miscarriage, spontaneous abortion or pre-eclampsia.

It is also envisaged that the administration of IFN-γ inhibitor in the presence or absence of the at least one surface antigen may need to continue past the prospective date of conception perhaps for the first 12 weeks of pregnancy.

The present invention may be used in conjunction with IVF treatment or other forms of assisted reproductive technology (ART), whereby the GM-CSF synthesis or transient tolerant immune response is elicited before transfer of the conceptus or gametes is attempted. It is expected however that where the reproductive disorder is a result of elevated IFN-γ levels in semen, it is likely that IVF treatment may not be needed, and that a “natural” conception may be possible in its place.

Alternatively the IFN-γ inhibitor and/or TGF-β protein may be administered to the reproductive tract of the female before, during and/or after intercourse. Thus the one or more antigens are delivered by the male ejaculate.

The invention also encompasses a vaginal cream formulation, which comprises an agent capable of inhibiting the binding of IFN-γ with the IFN-γ receptor in the female reproductive tract.

Treatment of Infertility Disorders

Specific disorders or procedures that may be treated using the methods described above include but are not limited to the following:

Recurrent miscarriage. It is known that approximately 2 -5% of couples are involuntarily childless due to recurrent miscarriage. The aetiology of recurrent miscarriage is not yet understood, but in the vast majority of cases no chromosomal, hormonal nor anatomical defect can be found and an immunological problem is implicated. A variety of therapies which attempt to modify the mother's immune response to the semi-allogeneic conceptus have been tested, with variable success. The predominant therapeutic approach over the past 20 years has been to inject women with paternal leucocytes in the hope of achieving ‘tolerance’ to paternal antigens. This therapy has had limited success, with a meta-analysis of 15 trials concluding that paternal leucocyte immunisation can increase pregnancy rates by 8-10% (Gleicher Am. J Reprod Immun. 32, 55-72 (1994)), and it is now regarded as being contra-indicated. The present invention discloses inter alia that decreasing IFN-γ levels is a beneficial treatment for recurrent miscarriage because of its inhibition of GM-CSF synthesis and potent immune modulating capacity. In particular, administration of sperm in combination with an IFN-γ inhibitor produces a tolerant or ‘nurturing’ immune response to a future conceptus which would share some of the same MHC class I or other antigens.

Adjunct to IVF treatmen or other assisted reproduction technologiest. It is currently believed that premenstrual pregnancy wastage produces a significant negative contribution to IVF success rates. One theory for this increased early pregnancy loss is that IVF is an “unnatural” process which separates the act of intercourse from conception. This would mean that IVF recipients may not be exposed to seminal plasma and its associated GM-CSF synthesis and tolerance inducing activity early in pregnancy. It has been suggested that exposure of the female genital tract to semen at the initiation of a pregnancy, as well as prior to a pregnancy, is beneficial to subsequent pregnancy outcome. The present invention comprehends treatment of women with an exogenous IFN-γ inhibitor in combination with partner's semen/leucocytes at or near the time of embryo transfer, especially if the partner's seminal plasma IFN-γ content is high or sperm numbers are low.

Anti-sperm antibody therapy. A significant proportion of infertility is due to the presence of anti-sperm antibodies in either the male or female partner (Kutten et al. Mol Androl 4, 183-193 (1992)). Seminal plasma has been shown to suppress the formation of anti-sperm antibodies in the female serum and genital tract secretions of the mouse. One of the active agents within seminal plasma which is responsible for suppressing maternal production of potentially damaging, complement-fixing isotypes or subclasses of immunoglobulin specific for sperm antigens has been identified as TGFβ. Since the effects of TGFβ are antagonised by IFN-γ, the methods of the present invention may be used to block anti-sperm antibody formation. Current therapies for anti-sperm antibodies are insufficiently effective, for example oral steroids or the prolonged use of barrier contraception, or require expensive and unpleasant assisted reproduction therapy. Administration of IFN-γ inhibitor before, during or following intercourse reduces this anti-sperm antibody response and enable natural pregnancy to ensue.

Pre-eclampsia and IUGR prophylaxis. Pre-eclampsia and some forms of intra-uterine growth restriction (IUGR) are believed to be an immunological disorder caused by “shallow” placentation resulting from damaging, type 1 immune attack on the invasive trophoblast. There is epidemiological evidence showing that repeated exposure of a woman to her partner's antigens through intercourse in the absence of barrier contraception decreases her chances of developing pre-eclampsia in a subsequent pregnancy to that partner (Klonoff-Cohen et al. JAMA. 262, 3143-3147 (1989); Robillard et al The Lancet 344, 973-975 (1995)). This may result from the generation of maternal ‘tolerance’ towards paternal antigens as a consequence of repeated exposure at intercourse, which facilitates placental growth and invasion of the maternal decidua. Some women have a propensity to develop pre-eclampsia or to suffer fetal growth restriction every time they become pregnant. This may be due to elevated IFN-γ content of their partner's semen.

Priming with partner's antigens in combination with an IFN-γ inhibitor before conception and perhaps until 3 months of pregnancy, by which time placental invasion is complete, helps to prevent the development of pre-eclampsia and IUGR in these high-risk women.

Prospective analysis of stud animal fertility in animal breeding. Variability in the productivity of stud males is a major constraint in pig, cattle, sheep, horse, goat and other livestock breeding programs. In many species there are substantial differences between studs, particularly in the pre-implantation mortality of embryos sired, even within a given herd. Currently, reliable estimation of the fertility and fecundity of a stud male is only possible after documentation of the outcome of multiple pregnancies. Measurement of the IFN-γ content of seminal plasma of potential studs, for example by simple enzyme-linked immunosorbent assay, is an effective tool in animal breeding management. This approach is also applicable to breeding programs for racehorses and for companion animal such as dogs and cats, and in zoo bresding breeding programs.

Optimisation of pregnancy outcome in animal breeding. A primary determinant of the productivity of animal breeding programs, particularly in species such as the pig where litters are large, is variability in the litter size and weight of offspring. As described above, these parameters are believed to be influenced largely by the extent to which the mother is ‘tolerized’ to paternal antigens shared by the conceptus, or by the amount of GM-CSF synthesis in early pregnancy. Pregnancy outcome is often further compromised where the pregnancy is initiated by artificial insemination, particularly when artificial semen extenders, as opposed to seminal plasma, are employed as the carrier. Since the frequency of mating between breeding females and studs is often limited, and since variability in the seminal plasma TGFβ and IFN-γ content between males is probable, pregnancy outcome in many animal species will benefit from exogenous administration of IFN-γ inhibitor iIFN-γ inhibitor may be given prior to, or at the initiation of a naturally-sired pregnancy, or at the time of artificial insemination.

The present invention, thus generally described, will be understood more readily by reference to the following examples, which are provided by way of illustration and are not intended to be limiting of the present invention.

### EXAMPLE 1

### Presence of IFN-γ in Human Seminal Plasma and Relationship with Fertility Status

The Aim of this Study was to Investigate the Relationship Between Semen Content of IFN-γ and Fertility Status.

Human Interferon Gamma ELISA

A quantitative human interferon gamma (IFN-γ)-specific sandwich ELISA (R & D Systems) was used to measure the IFN-γ content of human seminal plasma. Both the reagent preparation and the immunoassay were performed according to the manufacturer's instructions. A polyclonal antibody specific for IFN-γ was bound to 96 well microtitre plates in order to capture IFN-γ from recombinant standard or seminal plasma samples. Assay diluent (100 μl, RD1-51) was added to each of the wells followed by the addition of 100 μl of standard or seminal plasma. The plate was then covered with an adhesive strip and incubated at room temperature for 2 hours. Following incubation, the contents of the plate were aspirated and washed a total of 4 times with wash buffer (provided by manufacturer). The plate was then blotted against clean paper towelling to remove any residual wash buffer. 200 μl of IFN-γ-specific polyclonal antibody conjugated to horseradish peroxidase (HRP) was then added to each of the wells. The plate was covered with a fresh adhesive strip and incubated at room temperature for 2 hours. Following incubation, the plate was again washed as described previously. Bound antibody was then quantified by the addition of a HRP chromagen substrate. Substrate solution (200 μl) was added to each of the wells and incubated at room temperature for 30 minutes. Following incubation, the substrate was acidified by the addition of 50 μl of 2N sulphuric acid, and absorbance at 450 nm (wavelength correction set to 570 nm) was measured. The concentration of IFN-γ in human seminal plasma samples was calculated from a standard curve generated using known concentrations of rhIFN-γ. The manufacturer's assay specifications stated that the minimal quantifiable level of IFN-γ was 7.8 pg/ml (although IFN-γ was clearly detectable to approximately 5 pg/ml), with intra-assay variation of 4.7% and an inter-assay variation of 7.8%.

Human Semen Samples

Male subjects attending the University of Adelaide Reproductive Medicine Unit donated semen after providing informed consent and after institutional ethics approval was granted. Semen samples were produced by masturbation and processed within 30 minutes of collection. Routine semen analysis was performed to determine volume of semen, sperm concentration, sperm motility, sperm morphology and leukocyte content, and seminal plasma was obtained after removal of sperm and cellular debris by high speed centrifugation. Following centrifugation, the supernatant was divided into aliquots and stored at −70° C. until use.

Seminal plasma was obtained from men classified according to fertility status and semen characteristics into the following groups:

1. Fertile. This group includes semen from men with normal semen parameters according to WHO criteria and proven fertility as evidenced by one or more live born children.

2. Normal. This group includes semen with normal semen characteristics as defined by WHO criteria. Subjects included men of unknown fertility status (data on children fathered unavailable), as well as male partners of women diagnosed with female factor infertility eg. anovulation, tubal factors such as blocked fallopian tubes, and endometriosis.

3. Male infertility. This group includes men with any defect in the semen analysis below the reference ranges according to WHO criteria.

4. Combined male and female infertility. This group includes men with any defect in the semen analysis below the reference ranges according to who criteria, with female partners diagnosed with anovulation or blocked fallopian tubes.

5. Recurrent IVF failure. This group includes male partners of women who have undergone IVF treatment and failed to achieve a live birth despite transfer of 10 good quality embryos over 3 or more treatment cycles. Subjects were included in this group regardless of the concurrent presence of male or female factor infertility. They were not included in any other group.

6. Recurrent miscarriage of unknown origin. Subjects in this group included male partners of women who have experienced three or more miscarriages where no cause for miscarriage is identified. Male partners of RM women with known aetiology (eg genetic, thrombophilia, severe maternal disease including diabetes etc) are not included in this group. One male in this group was the partner of a woman with two identified miscarriages and additional suspected miscarriages after repeated IVF treatment with more than 15 viable embryos transferred.

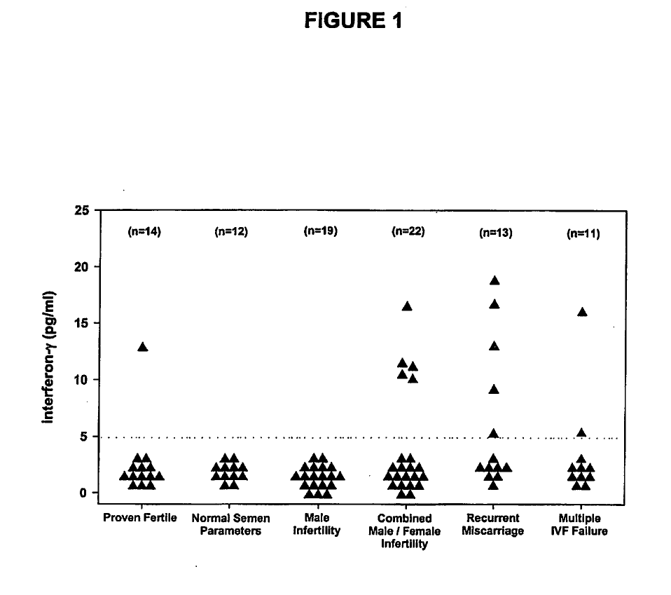

IFN-γ was detectable in the semen of 1 of 14 proven fertile men, and none of 12 additional men with normal sperm parameters These results are summarized in FIG. 1.

In contrast, IFN-γ was more frequently detected in the semen of male partners of infertile couples. Most notably, IFN-γ was detectable in semen of 5 of 13 male partners of women who experienced recurrent miscarriage, with a mean IFN-γ content in positive men of 12.5 pg/ml. This was despite the presence of normal sperm parameters in each of these men, including sperm concentration >20 million sperm/ml. One of 13 males tested positive for IFN-γ on each of three occasions (9.1, 6.6 and 8.7 pg/ml), when semen samples were provided at intervals separated by one-two over the course of a four month period.

IFN-γ was detectable in semen of 2 of 11 male partners of women with repeated IVF failure, and 5 of 22 male partners in couples where both male and female factors were implicated. IFN-γ was not detectable in the semen of any of 19 men with abnormal sperm parameters.

### EXAMPLE 2

### Effect of IFNγ, Penicillin and TGFβ on GM-CSF Synthesis in Murine Uterine Epithelial Cells

Uterine Epithelial Cell Cultures. Uterine epithelial cells prepared as previously described (Robertson et al., Biol Reprod 46:1069-1079 (1992)) were pooled from 4-6 estrous C57B1/6 or C57B1/6×Balb/c F1 mice and plated in 1 ml culture wells (Nunc, Roskilde, Germany) at 1-2×10⁵ cells per ml in 500 μl of Dulbecco's modified Eagles medium containing 10% FCS, 100 mg/ml streptomycin sulphate and 60 mg/ml benzyl penicillin (DMEM +penicillin). After 4 h incubation at 37° C. in 5% CO₂ to allow cell adherence, a further 500 μl of DMEM +penicillin, or DMEM +penicillin plus recombinant mouse IFNγ (rIFNγ) and/or recombinant human TGFβ1 (rTGFβ), were added. In some experiments, penicillin-free DMEM (DMEM −penicillin) was used throughout. Culture supernatants were collected and replaced with fresh medium at 16 hours, then collected again 24 hours later, at which time GM-CSF content was determined and adherent cells were quantified as previously described (Robertson et al., Biol Reprod 46:1069-1079 (1992)). All treatments were performed in duplicate or triplicate. rTGFβ and rIFNγ were obtained from R&D Systems, UK.

GM-CSF bioassay: GM-CSF was assayed using the GM-CSF dependant cell line FD 5/12, essentially as previously described (Robertson et al., Biol Reprod 46:1069-1079 (1992)). Cell proliferation was determined by pulsing with 1 μCi of [³H]-thymidine per well for the last 6 h of the assay. The minimal detectable amount of GM-CSF was 20 pg/ml. The identity of the bioactivity in uterine epithelial cell cultures was confirmed using a goat polyclonal antibody to murine GM-CSF (DNAX, Palo Alto, Calif.) as previously described (Robertson et al., Biol Reprod 46:1069-1079 (1992)). GM-CSF production is expressed as ng GM-CSF/10⁵ cells/24 h. Recombinant mouse GM-CSF was obtained from R&D Systems.

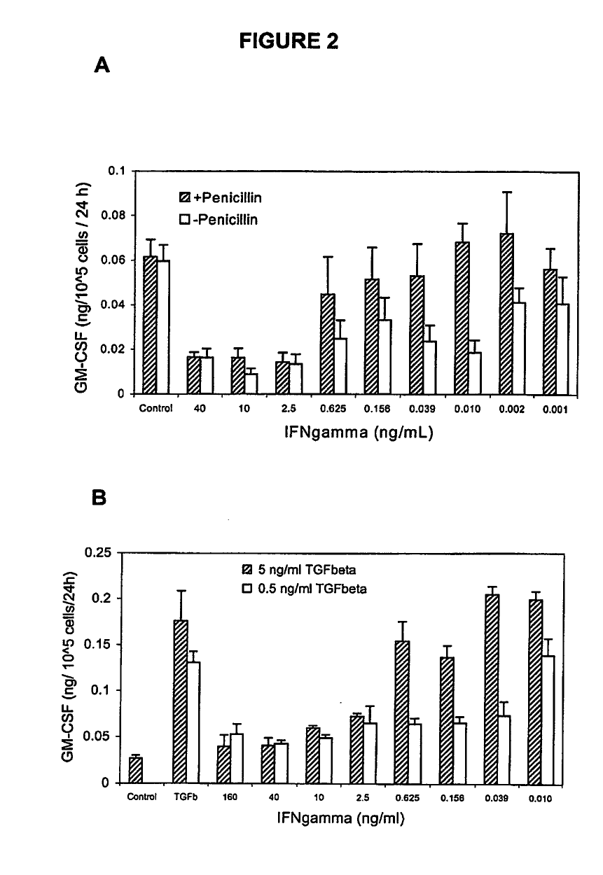

The results are shown in FIG. 2.

IFN-γ inhibits constitutive GM-CSF synthesis in uterine epithelial cells. To investigate the effect of IFN-γ on cytokine synthesis and the immune response in the female tract, uterine epithelial cells recovered from estrous mice were cultured in vitro in DMEM +penicillin with rIFN-γ. In each of three experiments a dose-dependent decrease in GM-CSF production was seen, with maximal decrease elicited at a concentration of approximately 2.5 ng/ml or higher of rIFN-γ (FIG. 2A: ‘+penicillin’). An inhibitory effect of rIFNγ was not seen at concentrations less than 150 pg/ml in experiments conducted in DMEM +penicillin.

Penicillin inhibits effect of IFN-γ in uterine epithelial cells. To investigate the ability of β-lactam antibiotics to inhibit the effect of IFN-γ in uterine epithelial cells, the inhibitory effect of IFNγ was examined in media from which penicillin was omitted (DMEM −penicillin). In each of three experiments a dose dependent decrease in GM-CSF production from was seen, with maximal decrease at a concentration of 2.5 ng/ml or higher of rIFNγ (FIG. 2A: ‘−penicillin’). In the absence of penicillin, the inhibitory effect of rIFNγ was maintained at lower concentrations than seen in the presence of penicillin, and observed at a concentration of 2 pg/ml of rIFNγ, attesting to an ability of penicillin to inhibit the effect of IFNγ in uterine epithelial cells.

IFN-γ inhibits TGFβ-stimulated GM-CSF synthesis in uterine epithelial cells. To examine the ability of IFNγ to interfere with the capacity of TGFβ to stimulate GM-CSF production from epithelial cells in vitro, rTGFβ was added at two concentrations (5 ng/ml and 0.5 ng/ml) to cultures of uterine epithelial cells harvested from estrous mice. These are concentrations shown previously to elicit maximal and submaximal GM-CSF production from epithelial cells in vitro (WO98/39021, Tremellen et al., Biology of Reproduction, 58, 1217-1225 (1998)). In both of two experiments conducted in DMEM +penicillin, rTGF, elicited an increase in GM-CSF production of approximately 6-fold at a concentration of 5 ng/ml. Addition of rIFNγ inhibited rTGFβ induction of GM-CSF synthesis in a dose-dependent manner, with maximal effects at concentrations of 2.5 ng/ml of rIFNγ or higher (FIG. 2B). A relationship between the concentration of rTGFβ and the concentration of rIFNγ was seen, such that the lowest effective inhibitory concentration of rIFNγ was 2.5 ng/ml in the presence of 5 ng/ml of rTGFβ, but the lowest effective concentration of rIFNγ was 39 pg/ml in the presence of 0.5 ng/ml rTGFβ. These findings attest to the interrelationship between TGFβ and IFNγ in eliciting GM-CSF synthesis in uterine epithelial cells.

### EXAMPLE 3

### Effect of IFNγ and Various Inhibitors of IFNγ on GM-CSF Synthesis in Murine Uterine Epithelial Cells

Several inhibitors and antagonists of IFNγ were tested for their ability to counteract the GM-CSF-inhibiting actions of IFNγ in epithelial cells.

Uterine Epithelial Cell Cultures. Uterine epithelial cells prepared by pancreatin-trypsin digest as previously described (Robertson et al., (1992) Biol. Reprod. 46:1069-1079) were pooled from 4-6 estrous adult C57B1/6×CBA F1 female mice and plated in 1 ml culture wells (Nunc, Roskilde, Germany) at 1-2×10⁵ cells per ml in 500 μl of RPMI containing 10% FCS and streptomycin (RPMI-FCS). Penicillin was omitted from culture media except where specified. After 4 h incubation at 37° C. in 5% CO₂ to allow cell adherence, a further 500 μl of cytokines or antibodies in RPMI-FCS, or RPMI-FCS alone, were added. Cytokine treatments included recombinant mouse interferon-γ (rmIFNγ; R&D Systems), recombinant human transforming growth factor β1 (rhTGFβ1, R&D Systems), rhTGFβ2 (R&D Systems), rhTGFβ3 (OSI Pharmaceuticals). Antibody treatments included goat anti-mouse IFNγ receptor 1 (αIFNγR; R&D Systems AF1026) and hamster anti-mouse IFNγ (αIFNγ; R&D Systems MAB4851). Culture supernatants were collected and replaced with fresh complete RPMI-FCS alone at 16 hours, then collected again 24 hours later, at which time adherent cells were quantified as previously described (Robertson et al., (1992) Biol. Reprod. 46:1069-1079). The GM-CSF content of 24 h supernatants was determined by GM-CSF bioassay or immunoassay. All treatments were performed in duplicate or triplicate.

GM-CSF bioassay and immunoassay: Bioactive GM-CSF was assayed using the GM-CSF dependant cell line FD 5/12, essentially as previously described (Robertson et al., (1992) Biol. Reprod. 46:1069-1079). Cell proliferation was determined by the addition of 1 μCi of [³H]-thymidine per well for the last 6 h of the assay. The minimal detectable amount of GM-CSF was 20 ng/ml The identity of the bioactivity in uterine epithelial cell cultures was confirmed using a goat polyclonal antibody to murine GM-CSF kindly provided by J. Schreurs (DNAX, Palo Alto, Calif.) as previously described (Robertson et al., (1992) Biol. Reprod. 46:1069-1079). GM-CSF immunoactivity was in some experiments measured by commercial mouse GM-CSF specific ELISA (R & D Systems) according to the manufacturer's instructions.

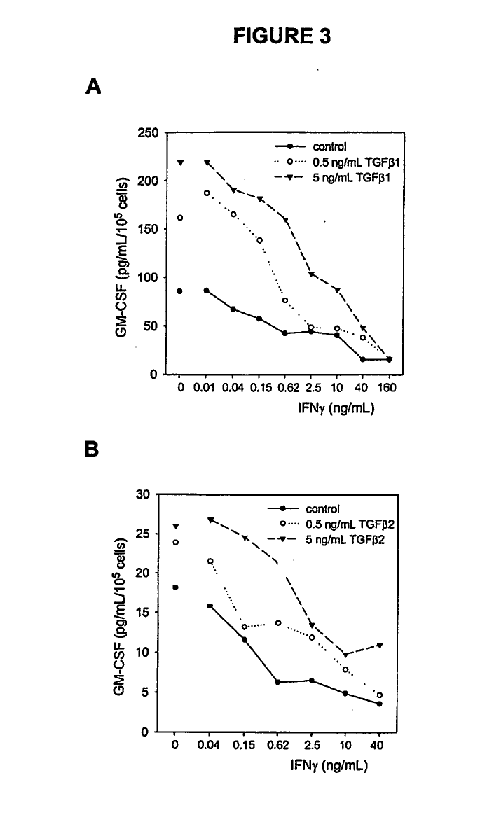

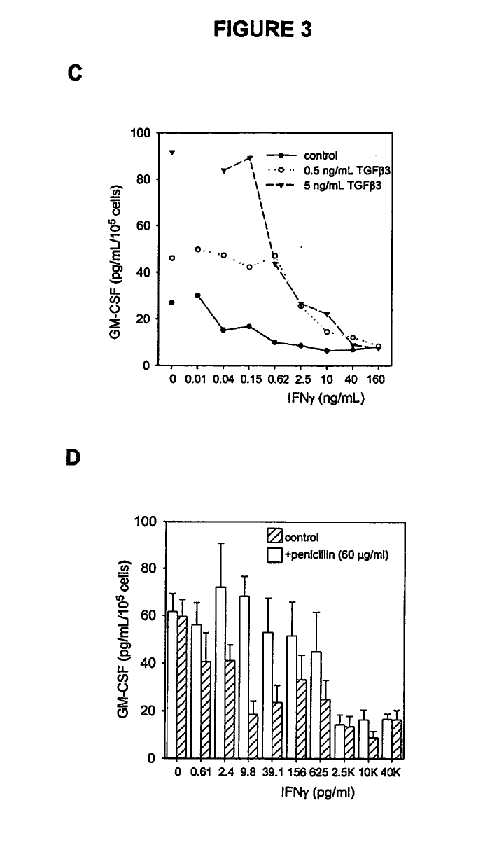

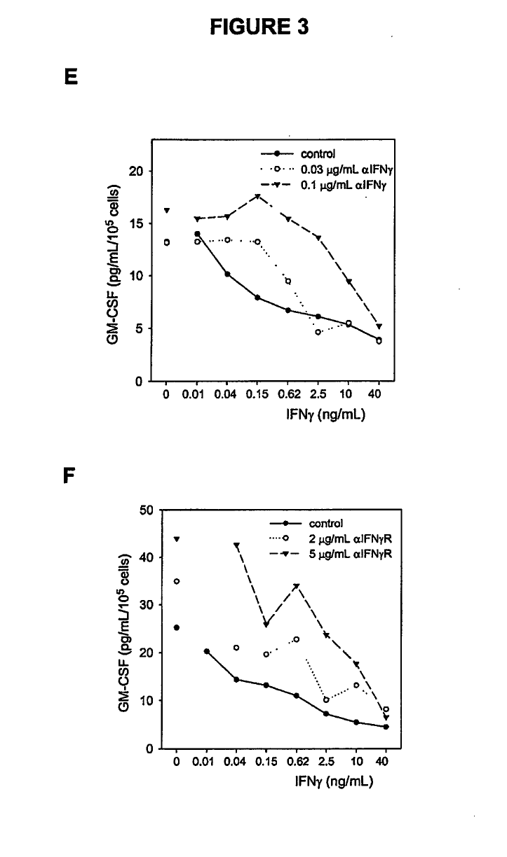

Addition of IFNγ to murine uterine epithelial cells was seen to inhibit GM-CSF synthesis in a dose responsive manner in each of more than six experiments (FIG. 3, A-F). Inhibition was evident at concentrations of IEFNγ of 40 pg/ml or greater. Significant inhibition (GM-CSF synthesis <50% of control values) was observed at concentrations of IFNγ>150 pg/ml.

Each of the three isoforms of TGFβ (rhTGFβ1, rhTGFβ2, and rhTGFβ3) when added in combination with IFNγ counteracted the inhibitory effect of this molecule, in a dose-responsive manner, as shown in FIG. 3A-C. Thus 5 ng/ml of each isoform of TGFβ had a greater effect than 0.5 ng/ml TGFβ. Addition of rhTGFβ1 or rhTGFβ3 at 0.5 ng/ml counteracted IFNγ up to concentrations of 620 pg/ml.

Addition of β-lactam penicillin, a known inhibitor of IFNγ, at 60 μg/ml also antagonised the GM-CSF inhibiting activity of IFNγ. In the absence of penicillin GM-CSF synthesis was inhibited (<50% of control values) at concentrations of IFNγ equal to or greater than 9.8 pg/ml, but addition of penicillin neutralised this effect such that concentrations of 2.5 ng/ml or more of IFNγ were required to inhibit GM-CSF synthesis, as shown in FIG. 3D.

Antibodies known to bind and neutralise IFNγ bioactivity also acted to antagonise the GM-CSF-inhibiting effect of this cytokine. At concentrations of 0.03 or 0.1 μg/ml, αIFNγ antibodies counteracted the effects of IFNγ at doses up to 150 pg/ml (FIG. 3E). Antibodies known to bind and the IFNγR1, a receptor entity required for IFNγ signalling in IFNγ responsive cells, also acted to antagonise the GM-CSF-inhibiting effect of IFNγ. At concentrations of 2 or 5 μg/ml, αIFNγR1 antibodies counteracted the effects of IFNγ at doses up to 620 pg/ml (FIG. 3F).

### EXAMPLE 4

### Effect of IFN-γ and TGFβ on GM-CSF Synthesis in Human Cervical Epithelial Cells

Human Cervical Epithelial Cells:

Human cervical epithelial cells were cultured using a modification of the technique described by Rheinwald et al., Cell 1975 3: 33143 (1975). Cervical biopsies were obtained from consenting women undergoing hysterectomy for non-malignant gynaecological indications. All the women were pre-menopausal, but no distinction was made regarding the stage of their menstrual cycle at the time of surgery. The cervical biopsies were placed in ice-cold Hank's balanced salt solution for transport to the laboratory, washed twice in antibiotic-containing medium, and incubated overnight at 4° C. in Dulbecco's modified Eagle's medium (DMEM) containing 5 U dispase (Boehringer Mannheim). Large sheets of keratinocytes were mechanically stripped from the biopsy using sterile forceps after a subsequent 1 h incubation at room temperature. Disaggregation into single cells was facilitated by incubation in DMEM/0.25% trypsin/0.05% collagenase for 30 minutes at 37° C., and repeated aspiration using a needle and syringe. Keratinocytes were cultured in ectocervical culture medium (ECM) consisting of 69% DMEM/23% Hams F-12/5% FCS/1% Nutridoma-SP (Boehringer Mannheim)/1% glutamine/1% hydrocortisone, at a density of 1-2×10⁵cells/ml, over monolayers of murine 3T3 fibroblasts which had been rendered mitogenically inactive by exposure to 4% mitomycin C (Sigma). Keratinocytes were incubated for 5-7 days to enable attachment and displacement of the 3T3 fibroblasts. Following incubation of the keratinocytes to enable attachment and displacement of the 3T3 fibroblasts, supernatant was collected 12 hours later to measure baseline levels of GM-CSF activity and replaced with

a) 500 μl of ECM-FCS containing 0.5 ng/ml, 5.0 ng/ml or 50 ng/ml of recombinant rTGFβ1 (R&D Systems),

b) 5.0 ng/ml of recombinant human IFN-γ (R & D Systems),

c) 0.5 ng/ml TGFβ1+5.0 ng/ml IFN-γ, 5.0 ng/ml TGFβ1+5.0 ng/ml IFN-γ, 50 ng/ml TGFβ1+5.0 ng/ml IFN-γ, or

d) culture medium only (control).

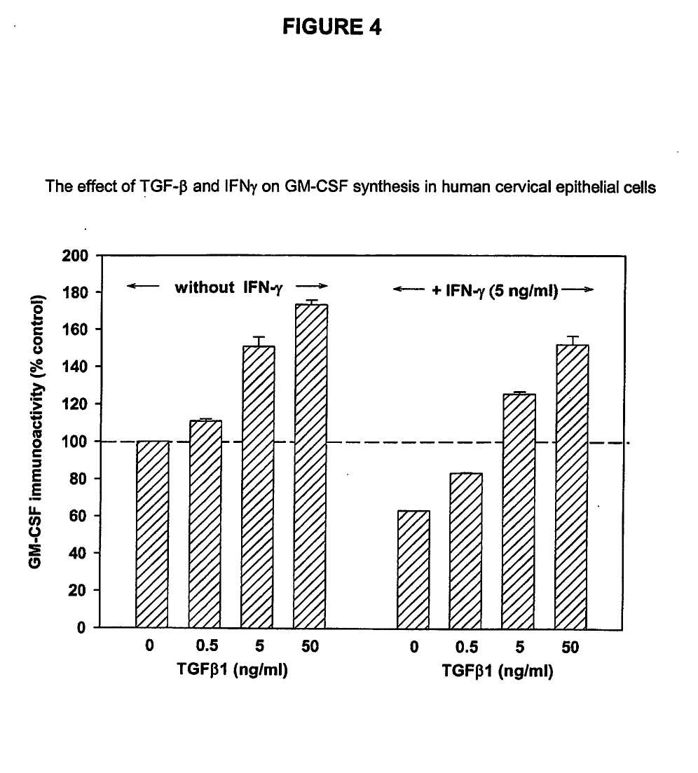

Supernatant was collected 12 hours later and assayed for GM-CSF activity using a commercial human GM-CSF specific ELISA (R & D Systems) according to the manufacturer's instructions. The GM-CSF content of post-treatment supernatants were normalised to the GM-CSF content of corresponding 12 h (baseline) supernatants and expressed as a percentage of control values. The results are shown in FIG. 4.

Addition of IFN-γ to human cervical epithelial cells inhibited GM-CSF synthesis. TGFβ added in combination with IFN-γ acted to counteract the inhibitory effect of this molecule, in a dose-responsive manner.

### EXAMPLE 5

### Expression of IFN-γ Receptor mRNA in Murine Uterine Epithelial Cells

Preparation of Mouse Uterine Epithelial mRNA and cDNA.

Uterine tissue was collected from estrous mice, and was either snap-frozen as whole uterus in liquid nitrogen and stored at −70° C., or treated to generate enriched uterine epithelial cells and residual uterine stromal cells. Uterine cells were purified according to a previously described protocol (Robertson et al., Biol Reprod 46:1069-1079 (1992)), using rat anti-mouse MTS#24 mAb specifically reactive with uterine epithelial cells for affinity purification of epithelial cells by “panning” from trypsin-pancreatin digested uterine cell suspensions. Purified uterine epithelial cells and residual stromal cells were snap-frozen in liquid nitrogen and stored at −70° C. Upon thawing, cells or tissue were homogenised in RNAzol B (Tel-Test, Friendswood, Tex.) and extracted with chloroform prior to precipitation of RNA from the aqueous phase in cold 97% EtOH at −20° C. RNA was treated with 10 U/μL DNase I (Roche, Basel, Switzerland) containing RNAse Inhibitor (Roche), then extracted in phenol-chloroform and precipitated in cold 97% EtOH. RNA was reverse transcribed using random hexamers (Geneworks, Adelaide, Australia) and Superscript II enzyme kit (Invitrogen, Carlsbad, Calif., USA) following the manufacturer's instructions.

Quantitative Real Time PCR.:

Primers for mRNAs encoding IFN-γ receptor (IFN-65 R), the TGFβ type 2 receptor (TGFβR2) and the “housekeeping” mRNA β-actin were designed using Primer Express (Applied Biosystems) and NCBI on-line facilities and were purchased from GENSET OLIGOs (Lismore, Australia). The sequences of these primers are set out in Table 2. Primers for “housekeeping” 18S mRNA were purchased from Ambion Austin, USA). PCR reactions were completed on an ABI Prism 5700 Sequence Detection System (Applied Biosystems). PCR reactions followed a three-stage program; 50° C. for 2 min; 95° C. for 10 min; and 40 cycles of (15 sec at 95° C. then 1 min at 60° C.). Raw data were analysed using the ΔΔCt method [User Bulletin #2, Applied Biosystems, ABI Prism 7700 Sequence Detection System, Livak and Schmittgen, 2001]. Following normalization of the amount of target gene to the content of β-actin mRNA, the value was multiplied by a constant (C) such that the average of the control value=100 mRNA Arbitrary Units (AU) using the equation 2^{ΔCt-β-Actin-ΔCt-Target}/C×100. All Ct values for a given primer set were multiplied by C to enable data expression in AU.

In addition, representative PCR products were electrophoresed on 2% agarose electrophoretic gels to assess amplicon size. Products were purified using Qiagen MinElute PCR Purification Kit (Clifton Hill, Victoria) and sequenced by Molecular Pathology (IMVS, Adelaide, Australia) to confirm sequence identity with target cDNA sequence.

TABLE 2

| | | | Genbank Accession |
|---|---|---|---|
| Target | Primer - Position | Amplicon size | Number |
| IFN-γR | Forward - 562 5′-AGAGAGCCAGGGAACCATGTT | 116bp | GI: 6652963 |
| | Reverse - 677 5′-TTTCGACCGTATGTTTCGTATGTAG | | |
| TGFβR2 | Forward - 377 5′-GCTGTGCAAGTTTTGCGATGT | 74bp | GI: 6678324 |
| | Reverse - 450 5′-TGATGCTGCAGTTGCTCATG | | |
| β-Actin | Forward - 182 5′-CCGTGGGCCGCCCTAGGCACCA | 170 bp | GI: 6671508 |
| | Reverse - 351 3′-CACGCAGCTCATTGTA | | |
| 18S | Universal Primers & Universal Competimer | 315 bp | Ambion |
| | Ambion, Austin, Texas, USA | | |

Quantitative real time RT-PCR reagents. Primer sequences, accession numbers of Genbank cDNA sequences and expected amplicon size are shown.

IFN-γ signals in target cells through ligation with its membrane-bound receptor IFN-γR. Each of the three isoforms of TGFβ signal in target cells through binding to membrane-bound TGFβ type I and type II receptors (TGFβR1 and TGFβR2 respectively).

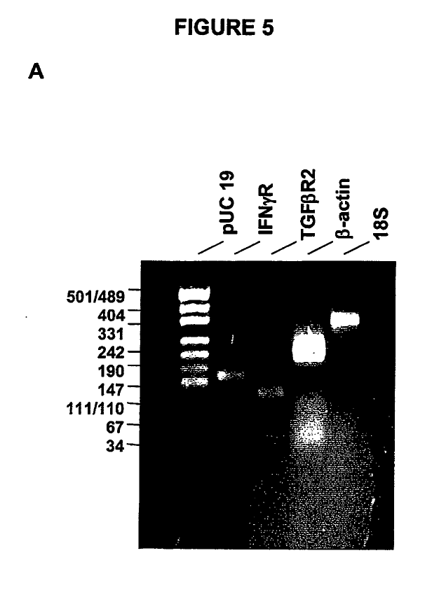

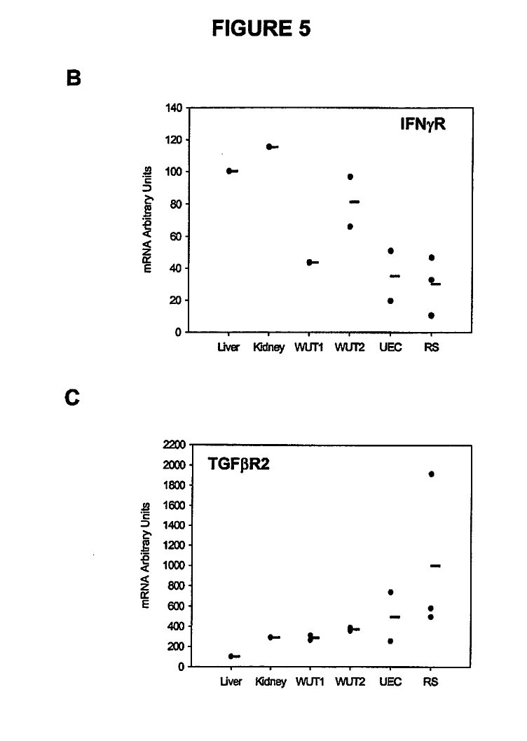

Messenger RNAs encoding IFN-γR and TGFβR2 were clearly detectable in two preparations of whole uterine tissues from estrous mice, as shown in FIG. 5A. PCR amplicons were of the expected size, and DNA sequencing confirmed their identity with the relevant cDNA templates. Uterine epithelial cells are amongst the cell lineages expressing IFN-γR in the uterus, since quantitative RT-PCR experiments in fractionated uterine cell preparations showed IFN-γR mRNA was present in comparable abundance in purified uterine epithelial cell preparations and residual stroma cells. Similarly, quantitative RT-PCR experiments in fractionated cells show that epithelial cells express TGFβR2 mRNA.

### EXAMPLE 6

### Expression of IFN-γ mRNA in Human Cervix

Preparation of Human Cervical Tissue mRNA and cDNA.

Tissue biopsies were collected using a Pipelle biopsy instrument from the ectocervix of n=17 proven fertile women 18-40 years of age at the Karolinska Hospital, Stockholm, Sweden, after obtaining ethics approval and informed consent. Sample recovery was timed to the time of ovulation (LH0-LH+1) using urinary LH testing, and women had not engaged in unprotected intercourse for one week prior to tissue biopsy. Tissue was snap-frozen in liquid nitrogen and stored at −70° C., and shipped to Adelaide on dry ice for processing. Upon thawing, tissue was homogenised in RNAzol B (Tel-Test, Friendswood, Tex.) and chloroform extracted prior to precipitation of RNA from the aqueous phase in cold 97% EtOH at −20° C. RNA was treated with 10 U/μL DNase I (Roche, Basel, Switzerland) containing RNAse Inhibitor (Roche), then extracted in phenol-chloroform and precipitated in cold 97% EtOH. RNA was reverse transcribed using random hexamers (Geneworks, Adelaide, Australia) and Superscript II enzyme kit (Invitrogen, Carlsbad, Calif., USA), following the manufacturers instructions.

Quantitative Real Time PCR: Primers for mRNAs encoding IFN-γ and the ‘housekeeping’ mRNAs β-actin were designed using Primer Express (Applied Biosystems) and NCBI on line facilities and were purchased from GENSET OLIGOs (Lismore, Australia). The sequences of the IFNα and β-active primers are shown in Table 3. Primers for “housekeeping” 18S mRNA were purchased from Ambion (Austin, Tex., USA). PCR reactions were completed on an ABI Prism 5700 Sequence Detection System (Applied Biosystems). PCR reactions followed a three stage program; 50° C. for 2 min; 95° C. for 10 min; and 40 cycles of (15 sec at 95° C. then 1 min at 60° C.). The mathematical tool for analysing raw data was the ΔΔCt method [User Bulletin #2, Applied Biosystems, ABI Prism 7700 Sequence Detection System, Livak and Schmittgen, 2001]. Following normalization of the amount of target gene to the content of β-actin mRNA, the value was multiplied by a constant (C) such that the average of the control value=100 mRNA Arbitrary Units (AU) using the equation 2^{ΔCt-β-Actin-ΔCt-Target}/C×100. All Ct values for a given primer set were multiplied by C to enable data expression in AU.

In addition, representative PCR products were electrophoresed on 2% agarose electrophoretic gels to assess amplicon size. Products were purified using Qiagen MinElute PCR Purification Kit (Clifton Hill, Victoria) and sequenced by Molecular Pathology (IMVS, Adelaide, Australia) to confirm sequence identity with target cDNA sequences.

TABLE 3

| | | | |
|---|---|---|---|
| | | Product | Genbank |
| Primer | Sequence | Size | Accession # |

| | | | |
|---|---|---|---|
| 18S | Universal Primers & Universal Competimer | 315 | |
| | Ambion, Austin, Texas, USA | | |
| β-actin | 5′-tgt gat ggt ggg tat ggg tc | 162 | M10277 |
| | 3′-aca cgc agc tca ttg ta | | |
| IFN-γ | 5′-gaa acg aga tga ctt cga aaa gct | 72 | M29383 |
| | 3′-tgt att gct ttg cgt tgg aca t | | |

Quantitative real time RT-PCR reagents. Primer sequences, accession numbers of Genbank cDNA sequences and expected amplicon size are shown.

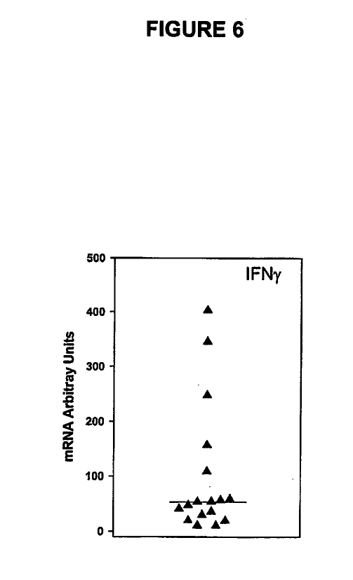

Messenger RNAs encoding IFN-γ were clearly detectable in each preparations of cervical tissue, and there was considerable variance in the abundance of transcripts, with expression in individual women ranging from 10% and up to 400% of the population mean value. These results are shown in FIG. 6. Comparable data were obtained regardless of whether data were normalised to β-actin or 18S housekeeping mRNAs. The PCR amplicons were of the expected size, and DNA sequencing confirmed their identity with the relevant cDNA templates.

### EXAMPLE 7

### Effect of IFN-γ Treatment on GM-CSF Content of Uterine Luminal Fluids in Mice

The aim of this experiment was to investigate the effect of IFN-γ on GM-CSF content of uterine luminal fluids after induction by exposure to semen on day 1 of pregnancy.

Mice and Treatments.

Mice were housed under specific pathogen-free conditions at the University of Adelaide Medical School Animal House on a 12:12 light-dark cycle, and were administered food and water ad libitum. Adult C57B1/6×CBA F1 females (7-10 weeks old) were caged with proven fertile Balb/c males for natural mating. The day of sighting a vaginal plug was nominated day 1 of pregnancy. Mice were randomly allocated to one of three groups and administered transcervical treatments of either 5 ng or 20 ng recombinant mouse IFN-γ (rmIFN-γ; R&D Systems) in 30 μl PBS containing 0.1% bovine serum albumin (PBS-BSA), or PBS-BSA alone (PBS control), at 0900 h on day 1. Treatments were delivered directly into the uterine lumen with the aid of a tom-cat catheter (Sovereign, St Louis Mo.), after removal of vaginal plugs using forceps to allow transcervical access. Mice were sacrificed 7 hrs later at 1600 h on day 1, and uterine luminal fluids were flushed in 1 ml PBS-BSA with a syringe and 26 gauge needle.

GM-CSF Bioassay and Immunoassay:

GM-CSF immunoactivity in uterine luminal fluids was measured by commercial mouse GM-CSF specific ELISA (R & D Systems) according to the manufacturer's instructions. Data are given as GM-CSF content in pg/ml uterine luminal fluid.

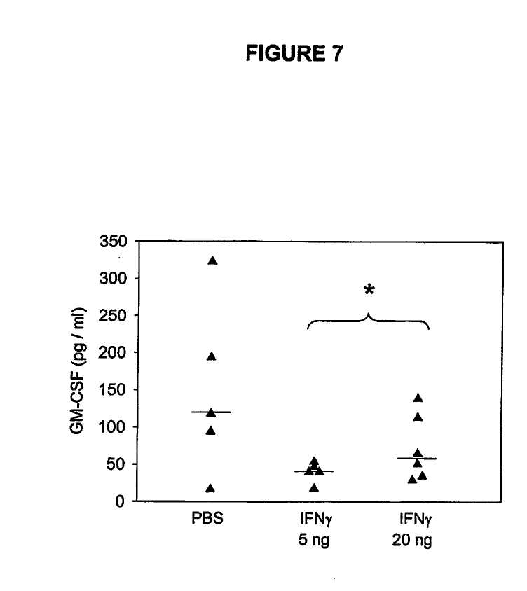

There was considerable variation in the GM-CSF content in uterine fluids in control animals, presumably reflecting variation in the amount of seminal fluid deposited in the uterine cavity. Application of either 5 ng or 20 ng rmIFN-γ was found to reduce uterine luminal fluid content of GM-CSF (p=0.03, PBS control group versus IFN-γ treatment groups combined). These results are presented in FIG. 7.

### EXAMPLE 8

### Effect of IFN-γ Treatment on Pregnancy Outcome in Mice

This example demonstrates the detrimental effect of administering exogenous recombinant IFN-γ, at the time of exposure to semen, on pregnancy outcome in mice.

Mice and Treatments.

Mice are housed under specific pathogen-free conditions at the University of Adelaide Medical School Animal House on a 12:12 light-dark cycle, and administered food and water ad libitum. Adult Balb/c×C57B1/6 F1 females (7-10 weeks old) are synchronised into estrus by caging in close proximity to males. Estrus is identified on the basis of presence of cornified epithelial cells in vaginal smears prepared at 0900-1000 h daily and examined by phase contrast microscopy. On the day of estrus (day 0) females are caged with proven fertile Balb/k males for natural mating. The day of sighting a vaginal plug is nominated day 1 of pregnancy.

Mice are randomly allocated to one of four groups and treated as follows: Group 1 (100 ng IFN-γ) mice receive three intraperitoneal injections of 50 ng recombinant mouse IFN-γ (rmIFN-γ, R&D Systems) in 200 μl PBS containing 1% bovine serum albumin (PBS-BSA) at 1700 h on day 0, at 1000 h on day 1 and 1700hon day 1.

Group 2 (250 ng IFN-γ) receive three injections of 250 ng rmIFN-γ according to the same time schedule.

Group 3 (control) receive three injections of PBS-BSA instead of IFN-γ according to the same time schedule.

Group 4 (comparison) Mice are mated but otherwise remain untreated.

Twenty mice per group are used to ensure statistical significance in treatment effects.

Alternatively, a transcervical delivery route for IFN-γ is used. Treatments are delivered directly into the uterine lumen with the aid of a tom-cat catheter (Sovereign, St Louis Mo.). In mated animals this requires removal of vaginal plugs using forceps to allow transcervical access. Mice are randomly allocated to one of four groups and treated as follows: the first group (100 ng IFN-γ) mice receive three transcervical injections of 5 ng recombinant mouse IFN-γ (rmIFN-γ, R&D Systems) in 200 μl PBS containing 1% bovine serum albumin (PBS-BSA) at 1700 h on day 0, at 1000 h on day 1 and 1700 h on day 1. The second group (25 ng IFN-γ) receive three injections of 25 ng rmIFN-γ according to the same time schedule. The third group (control) receive three injections of PBS-BSA instead of IFN-γ according to the same time schedule. For comparison, a fourth group of mice are mated but otherwise would remain untreated. Twenty mice per group are used to ensure statistical significance in treatment effects.

Pregnancy Outcome Measures:

Pregnant females are sacrificed by cervical dislocation at 1000 h -1200 h on day 18 of gestation. The intact uterus of each female is removed and total, viable and resorbing implantation sites counted. Each viable fetus is dissected from the amniotic sac and umbilical cord, and fetuses and placentae weighed. Placental tissues are fixed in paraformaldehyde and processed for histological staining of paraffin sections, using Masson's trichrome stain.

Administration of IFN-γ to the peritoneal cavity or alternatively to the uterine lumen on the day of estrus and day 1 of pregnancy impairs pregnancy outcome. A reduction in the overall implantation rate, and/or the proportion of viable versus resorbing fetuses on day 18, and/or the weight of the fetus and placenta on day 18 is observed. IFN-γ treatment results in changes in the fetal: placental weight ratio, and/or the structure of the placenta as assessed histologically, consistent with diminished placental function.

It is also found that administration of TGFβ (including TGFβ1, TGFβ2 or TGFβ3) or an IFN-γ inhibitor reverses the detrimental effects of IFN-γ and increases the pregnancy outcome parameters. In particular, administration of TGFβ (including TGFβ1, TGFβ2 or TGFβ3) or an IFN-γ inhibitor increases the overall implantation rate, and/or the proportion of viable versus resorbing fetuses on day 18, and/or the weight of the fetus and placenta on day 18.

Various features of the invention have been particularly shown and described in connection with the exemplified embodiments of the invention, however, it must be understood that these particular arrangements merely illustrate and that the invention is not limited thereto and can include various modifications falling within the spirit and scope of the invention.

1. A method of treating a reproductive disorder in a mammal, comprising administering an effective amount of a compound which inhibits the activity of IFN-γ to the mammal.

2. The method according to claim 1 wherein the mammal is a human.

3. A method according to claim 1, wherein the mammal is a prospective mother.

4. A method according to claim 1, wherein the mammal is a prospective father.

5. The method according to claim 1, wherein the compound binds to IFN-γ.

6. The method according to claim 5, wherein the compound is an antibody which binds specifically to IFN-γ.

7. The method according to claim 1, wherein the compound binds to the IFN-γ receptor.

8. The method according to claim 7, wherein the compound is an antibody which binds specifically to the IFN-γ receptor.

9. The method according to claim 5, wherein the compound is a soluble IFN-γ receptor.

10. The method according to claim 1, wherein the compound is selected from the group consisting of a β-lactam antibiotic, a protamine, a highly charged peptide, a highly charged protein, and a peptide comprising the sequence Arg-Arg-Lys-Trp-Gln (SEQ ID NO: 13)

11. The method according to claim 3, further comprising administering to the prospective mother an effective amount of a TGF-β.

12. The method according to claim 11, wherein the TGF-β is selected from the group consisting of TGFβ1, TGFβ2, TGFβ3, activin and analogues thereof.

13. The method according to claim 3, further comprising administering an effective amount of a GM-CSF to the prospective mother.

14. The method according to claim 1, wherein the reproductive disorder is selected from the group consisting of miscarriage, recurrent miscarriage, spontaneous abortion, pre-eclampsia, early embryonic loss, subfertility, and implantation failure.

15. The method according to claim 1, wherein the reproductive disorder is caused by a lack of immune tolerance to paternal antigen.

16. The method according to claim 15, wherein lack of immune tolerance is caused by the type 1 immune-deviating properties of IFN-γ.

17. The method according to claim 16, wherein the IFN-γ is present in the semen of a prospective father.

18. A method of treating a reproductive disorder in a mammalian prospective mother, comprising assaying the semen of a prospective father for the presence of IFN-γ and, if IFN-γ is detected, administering to the prospective mother an effective amount of a compound which inhibits the activity of IFN-γ.

19. The method according to claim 18, wherein the IFN-γ inhibitor is administered at the same time as the prospective mother is exposed to the semen of the prospective father.

20. The method according to claim 18, wherein the semen of the prospective father is treated with a compound which inhibits the activity of IFN-γ prior to delivery of the semen to the prospective mother.

21. The method according to claim 18, wherein the reproductive disorder is selected from the group consisting of miscarriage, recurrent miscarriage, spontaneous abortion, pre-eclampsia, early embryonic loss, subfertility, and implantation failure.

22. The method according to claim 18, wherein the reproductive disorder is caused by a lack of immune tolerance to paternal antigen.

23. The method according to claim 22, wherein lack of immune tolerance is caused by the type 1 immune-deviating properties of IFN-γ.

24. A method of treating a reproductive disorder in a mammalian prospective mother, comprising administering to a prospective mother an effective amount of a TGF-β, wherein the TGF-β composition is not TGFβ1, TGFβ2, TGFβ3, or activin.

25. The method according to claim 24, wherein the reproductive disorder is selected from the group consisting of miscarriage, recurrent miscarriage, spontaneous abortion, pre-eclampsia, early embryonic loss, subfertility, and implantation failure.

26. The method according to claim 24, wherein the reproductive disorder is caused by a lack of immune tolerance to paternal antigen.

27. The method according to claim 26, wherein lack of immune tolerance is caused by the type 1 immune-deviating properties of IFN-γ.

28. A method of treating a reproductive disorder in a mammalian prospective mother, comprising assaying the semen of a prospective father for the presence of IFN-γ and, if IFN-γ is detected, administering to the prospective mother an effective amount of TGF-β.

29. The method according to claim 28, wherein the TGF-P is selected from the group consisting of TGFβ1, TGFβ2, TGFβ3, activin and analogues thereof.

30. The method according to claim 28, wherein the TGF-β is administered via a mucosal surface.

31. The method according to claim 28, further comprising exposing the prospective mother to one or more antigens of a prospective father.

32. The method according to claim 31, wherein the one or more antigens are sperm antigens or MHC class I antigens present on leukocytes or in seminal plasma of the prospective father or a derivative or analogue of such antigen which comprises an epitope of the antigen.

33. The method according to claim 28, wherein the semen of a prospective father is treated with a compound which inhibits the activity of IFN-γ prior to delivery of the semen to the prospective mother.

34. The method according to claim 31, wherein the prospective mother is exposed to one or more antigens of the prospective father via a mucosal surface.

35. The method according to claim 31, wherein the prospective mother is exposed to one or more antigens of the prospective father before, during or after treatment with TGF-β.

36. The method according to claim 28, wherein the TGF-β composition is administered to the prospective mother prior to conception.

37. The method according to claim 31, in which immune tolerance to the one or more antigens is induced.

38. The method according to claim 28, wherein the reproductive disorder is selected from the group consisting of miscarriage, recurrent miscarriage, spontaneous abortion, pre-eclampsia, early embryonic loss, subfertility, and implantation failure.

39. The method according to claim 28, wherein the reproductive disorder is caused by a lack of immune tolerance to paternal antigen.

40. The method according to claim 39, wherein lack of immune tolerance is caused by the type 1 immune-deviating properties of IFN-γ.

41. A method of diagnosing a reproductive disorder in a mammalian prospective father, comprising comparing the amount of detectable IFN-γ in a semen sample obtained from the prospective father with a control sample, wherein a IFN-γ value higher than the control sample is indicative of the reproductive disorder.

42. The method according to claim 41, wherein the control sample is semen of a male partner of a female who does not suffer from a reproductive disorder caused by a lack of immune tolerance to paternal antigen.

43. The method according to claim 41, wherein the IFN-γ is detectable by ELISA.

44. The method according to claim 43, wherein the detectable level of IFN-γ is equal to or greater than 1 pg/ml.

45. A composition for treatment of a reproductive disorder, the composition comprising semen of a prospective father, together with either or both of
(a) an inhibitor of IFN-γ, and
(b) a member of the TGF-β family;
and optionally also comprising a pharmaceutically-acceptable carrier.

46. A composition according to claim 45, which is a vaginal cream, tampon or pessary.

47. A composition for treatment of a reproductive disorder, comprising
(a) a sperm or MHC Class I antigen of a prospective father,
(b) an inhibitor of IFN-γ, and/or
(c) a member of the TGF-β family, and
(d) a pharmaceutically-acceptable carrier.

48. A composition according to claim 47, which is a vaginal cream, tampon or pessary.

49. Use of a compound which inhibits the activity of IFN-γ for the manufacture of a medicament for the treatment of a reproductive disorder in a mammal.

50. Use according to claim 49, in conjunction with use of
a) a TGFβ,
b) GM-CSF, or
c) a sperm antigen or MHC Class I antigen present on leukocytes or in seminal plasma of a prospective father or a derivative or analogue of such an antigen which comprises an epitope of the antigen.

51. A kit for the diagnosis of a reproductive disorder in a couple, the kit comprising a reagent for the detection and/or measurement of IFN-γ in semen of a prospective father of the couple, and/or comprising a reagent for the detection and/or measurement of IFN-γ in a tissue or body fluid sample of a prospective mother of the couple.


SEQUENCE LISTING (13 sequences)

SEQ ID NO: 1 - protein, 112 aa, Homo sapiens [sequence omitted]

SEQ ID NO: 2 - DNA, 339 bp, Homo sapiens
gctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga

SEQ ID NO: 3 - DNA, 21 bp, Artificial Sequence
  Forward Primer Sequence - Target IFN-Gamma-R
agagagccagggaaccatgtt

SEQ ID NO: 4 - DNA, 25 bp, Artificial Sequence
  Reverse Primer Sequence - Target IFN-Gamma-R
tttcgaccgtatgtttcgtatgtag

SEQ ID NO: 5 - DNA, 21 bp, Artificial Sequence
  Forward Primer Sequence - Target TGF-betaR2
gctgtgcaagttttgcgatgt

SEQ ID NO: 6 - DNA, 20 bp, Artificial Sequence
  Reverse Primer Sequence - Target TGF-betaR2
tgatgctgcagttgctcatg

SEQ ID NO: 7 - DNA, 22 bp, Artificial Sequence
  Forward Primer Sequence - Target beta-Actin
ccgtgggccgccctaggcacca

SEQ ID NO: 8 - DNA, 16 bp, Artificial Sequence
  Reverse Primer Sequence - Target beta-Actin
atgttactcgacgcac

SEQ ID NO: 9 - DNA, 20 bp, Artificial Sequence
  Forward Primer Sequence - Target beta-actin
tgtgatggtgggtatgggtc

SEQ ID NO: 10 - DNA, 17 bp, Artificial Sequence
  Reverse Primer Sequence - Target beta-actin
atgttactcgacgcaca

SEQ ID NO: 11 - DNA, 24 bp, Artificial Sequence
  Forward Primer Sequence - Target IFN-gamma
gaaacgagatgacttcgaaaagct

SEQ ID NO: 12 - DNA, 22 bp, Artificial Sequence
  Reverse Primer Sequence - Target IFN-gamma
tacaggttgcgtttcgttatgt

SEQ ID NO: 13 - protein, 5 aa, Unknown
  Peptide which inhibits the activity of IFN-gamma to a mammal
RRKWQ